(19) United States
(12) Patent Application Publication
(10) Pub. No.: US 2021/0265048 A1
(43) Pub. Date: Aug. 26, 2021

(54) DEVICE IMPLEMENTED PATIENT TRANSFER COORDINATION AND COMMUNICATIONS

(71) Applicant: TINUUM, LLC, Richfield, UT (US)

(72) Inventor: Stacy EKKER, Richfield, UT (US)

(21) Appl. No.: 17/177,089

(22) Filed: Feb. 16, 2021

Related U.S. Application Data
(60) Provisional application No. 62/979,663, filed on Feb. 21, 2020.

(51) Int. Cl. G16H 40/20; G16H 10/60; G16H 40/67; G16H 80/00; G16H 50/20; G16H 15/00; G16H 30/20; H04L 29/06; A61B 5/1171

(52) U.S. Cl. CPC G16H 40/20; G16H 10/60; G16H 40/67; G16H 80/00; A61B 5/1176; G16H 15/00; G16H 30/20; H04L 63/0861; H04L 63/083; G16H 50/20

(57) ABSTRACT

Methods, systems, and devices for healthcare communications and more specifically to medical provider to medical provider communications are described. A medical provider may access patient information associated with a patient via an application on a device. The medical provider may select, via the application, a remote medical provider from a list of remote medical providers or a medical diagnosis for the patient, or both, based on the patient information. The device may establish, using a communication protocol, a multimedia session with a remote device associated with the remote medical provider. The medical provider or the remote medical provider, or both may determine whether to initiate a patient transfer procedure for the patient based on the multimedia session. As a result, the medical provider may via the device transmit, to the remote device, an indication of whether to initiate the patient transfer procedure for the patient.

### CROSS REFERENCE

The present application for patent claims the benefit of U.S. Provisional Patent Application No. 62/979,663 by EKKER et al., entitled “DEVICE IMPLEMENTED PATIENT TRANSFER COORDINATION AND COMMUNICATIONS,” filed Feb. 21, 2020, assigned to the assignee hereof, and expressly incorporated by reference herein.

### FIELD OF TECHNOLOGY

The following relates generally to healthcare communications and more specifically to medical provider to medical provider communications and device implemented patient transfer coordination and communications.

### BACKGROUND

Some healthcare systems are deployed to provide various types of communication content such as voice, video, packet data, messaging, broadcast, and so on. Some healthcare systems are deployed in various healthcare environments to provide various types of healthcare communication related to the exchange of healthcare information, for example, such as between medical providers.

### SUMMARY

Some healthcare systems may be capable of supporting communication between various personnel at several locations through communication connections (e.g., via wired or wireless communications). For example, a medical provider responsible for providing care for patients may often utilize a communications network to communicate and exchange healthcare-related information with other medical providers. As demand for efficiency, reliability and latency in healthcare environments increases, some healthcare systems may be unable to meet efficiency, reliability and latency metrics due to limitations in healthcare system infrastructure, a lack of organization or integration between medical providers, staffing capabilities, human error, and the like. Therefore, an improvement in healthcare systems, such as providing robust and efficient healthcare communications between medical providers may be desired.

Various aspects of the described techniques relate to healthcare communications and more specifically to medical provider to medical provider communications. For example, the described techniques may provide for communication and exchange of healthcare-related information (also referred to as patient information) between a medical provider and another medical provider to deliver care (e.g., a specialty care) for a patient. In some examples, a local medical provider may establish a multimedia session (e.g., audio communication, video communication) with a remote medical provider. As part of the multimedia session, the local medical provider may provide patient information (e.g., a medical diagnosis, information associated with the medical diagnosis) to the remote medical provider. In some examples, based on the multimedia session (e.g., based on a user input associated with the multimedia session), the local medical provider or the remote medical provider, or both, may initiate a patient transfer procedure for the patient. As such, the described techniques may improve efficiency, reliability and latency in healthcare environments, and more particularly for transferring patients between medical providers, thereby reducing latency involved in patient transfer procedures and ensuring that patients are provided appropriate care based on medical diagnoses.

A method for communications is described. The method may include accessing patient information associated with a patient via an application on a device, selecting, via the application, a remote medical provider from a list of remote medical providers or a medical diagnosis for the patient, or both, based on the patient information, establishing, using a communication protocol, a multimedia session between the device and a remote device associated with the remote medical provider, determining whether to initiate a patient transfer procedure for the patient based on the multimedia session, and transmitting, to the remote device, an indication of determining whether to initiate the patient transfer procedure for the patient.

An apparatus for communications is described. The apparatus may include a processor, memory coupled with the processor, and instructions stored in the memory. The instructions may be executable by the processor to cause the apparatus to access patient information associated with a patient via an application on the apparatus, select, via the application, a remote medical provider from a list of remote medical providers or a medical diagnosis for the patient, or both, based on the patient information, establish, using a communication protocol, a multimedia session between the apparatus and a remote apparatus associated with the remote medical provider, determine whether to initiate a patient transfer procedure for the patient based on the multimedia session, and transmit, to the remote apparatus, an indication of determining whether to initiate the patient transfer procedure for the patient.

Another apparatus for communications is described. The apparatus may include means for accessing patient information associated with a patient via an application on the apparatus, selecting, via the application, a remote medical provider from a list of remote medical providers or a medical diagnosis for the patient, or both, based on the patient information, establishing, using a communication protocol, a multimedia session between the apparatus and a remote apparatus associated with the remote medical provider, determining whether to initiate a patient transfer procedure for the patient based on the multimedia session, and transmitting, to the remote apparatus, an indication of determining whether to initiate the patient transfer procedure for the patient.

A non-transitory computer-readable medium storing code for communications is described. The code may include instructions executable by a processor to access patient information associated with a patient via an application on a device, select, via the application, a remote medical provider from a list of remote medical providers or a medical diagnosis for the patient, or both, based on the patient information, establish, using a communication protocol, a multimedia session between the device and a remote device associated with the remote medical provider, determine whether to initiate a patient transfer procedure for the patient based on the multimedia session, and transmit, to the remote device, an indication of determining whether to initiate the patient transfer procedure for the patient.

Some examples of the method, apparatuses, and non-transitory computer-readable medium described herein may further include operations, features, means, or instructions for terminating the multimedia session, and providing, via an interface of the application, a selectable option to consult the patient at a local medical provider or transfer the patient to the remote medical provider based on terminating the multimedia session.

Some examples of the method, apparatuses, and non-transitory computer-readable medium described herein may further include operations, features, means, or instructions for receiving an input associated with the selectable option, where the input indicates a selection to transfer the patient to the remote medical provider, initiating the patient transfer procedure based on the selection to transfer the patient to the remote medical provider. In some aspects, transmitting, to the remote device, the indication may be based on the initiating.

In some examples of the method, apparatuses, and non-transitory computer-readable medium described herein, the patient transfer procedure may include operations, features, means, or instructions for transmitting a patient transfer notification to the remote device associated with the remote medical provider, the patient transfer notification including the patient information, and receiving, at the device, an acknowledgment to transfer the patient to the remote medical provider based on the patient transfer notification.

Some examples of the method, apparatuses, and non-transitory computer-readable medium described herein may further include operations, features, means, or instructions for receiving, from the remote device associated with the remote medical provider, patient transfer information including physician information, room information, additional medical personnel information, or any combination thereof.

Some examples of the method, apparatuses, and non-transitory computer-readable medium described herein may further include operations, features, means, or instructions for receiving an input associated with the selectable option, where the input indicates a selection to consult the patient at the local medical provider, transcribing the multimedia session based on the selection to consult the patient at the local medical provider, and providing a report of the transcribed multimedia session to the local medical provider or the remote medical provider, or both.

Some examples of the method, apparatuses, and non-transitory computer-readable medium described herein may further include operations, features, means, or instructions for transmitting, to the remote device associated with the remote medical provider, a request message to establish the multimedia session, the request message including session information for the multimedia session, and receiving, from the remote device associated with the remote medical provider, a response message based on the request message, the response message including a second indication of accepting, delaying, or declining the request message to establish the multimedia session, the session information including the patient information or the medical diagnosis for the patient, or both. In some aspects, establishing the multimedia session between the device and the remote device may be based on the response message.

Some examples of the method, apparatuses, and non-transitory computer-readable medium described herein may further include operations, features, means, or instructions for retrieving one or more images corresponding to the medical diagnosis for the patient, and including, in the session information, the one or more images corresponding to the medical diagnosis for the patient.

In some examples of the method, apparatuses, and non-transitory computer-readable medium described herein, the multimedia session includes an audio session or a video session, or both.

Some examples of the method, apparatuses, and non-transitory computer-readable medium described herein may further include operations, features, means, or instructions for determining the list of remote medical providers based on the patient information or the medical diagnosis for the patient, or both, and determining one or more locations associated with one or more remote medical providers, including the remote medical provider, in the list of remote medical providers that satisfy a location threshold relative to the patient. In some aspects, selecting the remote medical provider from the list of remote medical providers may be based on the location threshold relative to the patient.

Some examples of the method, apparatuses, and non-transitory computer-readable medium described herein may further include operations, features, means, or instructions for determining the medical diagnosis for the patient based on the patient information, and including a second indication of the medical diagnosis in session information for the multimedia session.

Some examples of the method, apparatuses, and non-transitory computer-readable medium described herein may further include operations, features, means, or instructions for identifying a patient account corresponding to the patient, where accessing the patient information includes retrieving the patient information from the patient account via the application running on the device or a server, or any combination thereof.

Some examples of the method, apparatuses, and non-transitory computer-readable medium described herein may further include operations, features, means, or instructions for capturing an image of a document including the patient information via a camera of the device, determining one or more information elements of the document using an optical character recognition operation on the document, and compiling the patient information based on the one or more information elements.

Some examples of the method, apparatuses, and non-transitory computer-readable medium described herein may further include operations, features, means, or instructions for generating a patient account corresponding to the patient associated with the patient information, and storing the patient account in a database remote or local to the device or a server, or any combination thereof, where accessing the patient information includes retrieving the patient information from the patient account via the application running on the device or the server, or any combination thereof.

Some examples of the method, apparatuses, and non-transitory computer-readable medium described herein may further include operations, features, means, or instructions for determining a request to access the application running on the device, and providing the access to the application running on the device based on an authentication procedure.

In some examples of the method, apparatuses, and non-transitory computer-readable medium described herein, the authentication procedure may include operations, features, means, or instructions for receiving credential information via the device, the credential information including a username and a password, a facial recognition, a voice recognition, a biosignature recognition, or any combination thereof, and authenticating the device based on the credential information.

In some examples of the method, apparatuses, and non-transitory computer-readable medium described herein, the authentication procedure may include operations, features, means, or instructions for receiving first credential information from via the device, authenticating the first credential information, receiving second credential information via the device based on authenticating the first credential information, and authenticating the second credential information, where the first credential information or the second credential information, or both includes a username and a password, a facial recognition, a voice recognition, a biosignature recognition, or any combination thereof.

In some examples of the method, apparatuses, and non-transitory computer-readable medium described herein, the medical diagnosis for the patient includes a cardiological medical diagnosis, a neurological medical diagnosis, a trauma medical diagnosis, or a triage medical diagnosis, or any combination thereof.

### BRIEF DESCRIPTION OF THE DRAWINGS

FIGS. 1 and 2 illustrate examples of systems that support medical provider to medical provider communications in accordance with aspects of the present disclosure.

FIGS. 3A through 3T illustrate examples of devices that support medical provider to medical provider communications in accordance with aspects of the present disclosure.

FIGS. 4A through 4H illustrate examples of devices that support medical provider to medical provider communications in accordance with aspects of the present disclosure.

FIGS. 5A through 5E illustrate examples of devices that support medical provider to medical provider communications in accordance with aspects of the present disclosure.

FIGS. 6 and 7 show block diagrams of devices that support medical provider to medical provider communications in accordance with aspects of the present disclosure.

FIG. 8 shows a block diagram of a communications manager that supports medical provider to medical provider communications in accordance with aspects of the present disclosure.

FIG. 9 shows a diagram of a system including a device that supports medical provider to medical provider communications in accordance with aspects of the present disclosure.

FIGS. 10 through 13 show flowcharts illustrating methods that support medical provider to medical provider communications in accordance with aspects of the present disclosure.

### DETAILED DESCRIPTION

Some healthcare systems may be widely deployed to provide exchange of patient information between medical providers. These healthcare systems may be, however, unable to meet efficiency, reliability and latency metrics due to limitations in healthcare system infrastructure at each medical provider. Other inefficiencies may include a deficiency of organization or system integration between medical providers that may impede efficient exchange of patient information for treating patients. For example, a physician treating a patient at one medical facility may seek to consult a specialist physician at another medical facility regarding the patient. In cases of lacking organization and system integration, the physician may experience delay in consulting the specialist physician and, as a result, the patient may experience delays in receiving appropriate care for a medical diagnosis. Thus, some healthcare systems (e.g., medical facilities, medical staff, etc.) may negatively impact patient care.

The described techniques relate to improved methods, systems, devices, and apparatuses for medical provider to medical provider communication that support communication techniques and the exchange of information between a medical provider and another medical provider, for example, for effectively and efficiently securing patient care (e.g., a specialty care) for a patient. For example, the described techniques may provide for a treating physician at a medical facility to seek consultation (e.g., via a remote consultation with a physician specialist located at another medical facility) when applicable for treating a patient. Based on the consultation, the treating physician may effectively determine whether to treat (e.g., consult) the patient on-site or transfer the patient to the other medical facility (e.g., for life saving measures).

Examples of aspects of devices provided according to the described techniques may provide for a physician (e.g., a specialty physician at a medical provider) to provide remote consultation and accept patient transfers. In some examples, medical staff (e.g., a nursing supervisor at a medical provider) may view patient information associated with requested patient transfers, and in some examples, may accept or deny the transfer requests. According to examples of aspects described herein, time critical illnesses that may benefit from specialty care, such as cardiology, neurology and trauma may be facilitated in an efficient and effective manner.

Particular aspects of the subject matter described herein may be implemented to realize one or more advantages. The described methods, systems, devices, and apparatuses provide techniques which may support medical provider to medical provider communications, among other advantages. As such, supported techniques may include features for establishing a multimedia session (e.g., an audio conference, a video conference, or both) between medical personnel at various medical facilities. The techniques may provide for effective determination of whether to initiate a patient transfer procedure based on the multimedia session, resulting in patient treatment at a location closer to the patient's home (e.g., closer to a local medical provider treating the patient), or at a specialist at another medical facility when additional care is applicable. Additionally, the improved techniques provide for effective communication of a patient transfer requests, a patient transfer confirmation, and details associated with the patient transfer, thereby reducing latency (e.g. time) and overhead related to initiating and securing a patient transfer.

Aspects of the disclosure are initially described in the context of wireless communications systems. Aspects of the disclosure are further illustrated by and described with reference to apparatus diagrams, system diagrams, and flowcharts that relate to medical provider to medical provider communications.

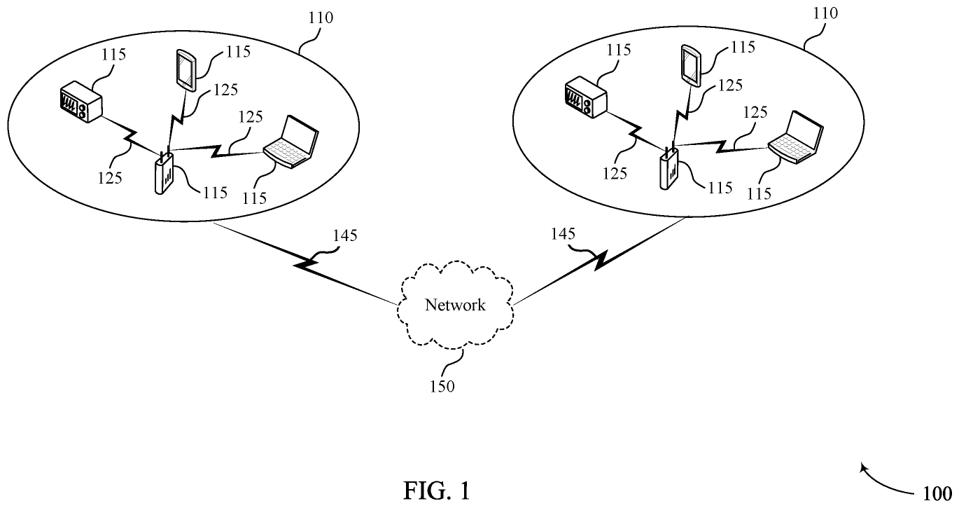

FIG. 1 illustrates an example of a system 100 that supports medical provider to medical provider communications in accordance with aspects of the present disclosure. The system 100 may include one or more medical facilities 110 (also referred to as medical providers or healthcare environments). For example, the system 100 may include three or more medical facilities 110. A medical facility 110 may be, in some examples, a hospital, a physician office, an urgent care clinic, a nursing home, and the like. Each medical facility 110 may include one or more devices 115. The devices 115 may be dispersed throughout the medical facility 110 and may be devices in different forms or having different capabilities. In some examples, the system 100 may support be a Long Term Evolution (LTE) network, an LTE-Advanced (LTE-A) network, an LTE-A Pro network, or a New Radio (NR) network. The devices 115 may wirelessly communicate within a medical facility via one or more communication links 125, or wirelessly communicate with other devices 115 at a remote medical facility 110 via one or more communication links 145 through a network 150, to support medical provider to medical provider communications. The network 150 may support one or more radio access technologies (e.g., via an LTE network, an LTE-A network, an NR network).

A device 115 may include or may be referred to as a mobile device, a wireless device, a remote device, a handheld device, or a subscriber device, or some other suitable terminology, where the “device” may also be referred to as a unit, a station, a terminal, or a client, among other examples. A device 115 may also include or may be referred to as a personal electronic device such as a cellular phone, a personal digital assistant (PDA), a tablet computer, a laptop computer, or a personal computer. In some examples, a device 115 may also be able to communicate directly with other devices 115 over a device-to-device (D2D) communication link (e.g., using a peer-to-peer (P2P) or D2D protocol). One or more devices 115 utilizing D2D communications may be within a medical facility 100. Other devices 115 in such a group may be outside a medical facility 110. In some examples, groups of the devices 115 communicating via D2D communications may utilize a one-to-many (1:M) system in which each device 115 transmits to every other device 115 in the group.

In an example, a device 115 may be associated with a personnel (e.g., a physician, a hospitalist, a nursing supervisor, a medical staff) at a medical facility 110 (e.g., a medical care facility, for example, a hospital, a clinic). According to examples of aspects described herein, the personnel may, using the device 115, access patient information associated with a patient via an application on the device 115. The device 115 may select, via the application, a remote medical provider (e.g., a medical care facility, for example, a hospital) from a list of remote medical providers, based on the patient information. In some examples, the personnel may use the device 115 to select a medical diagnosis for the patient.

The personnel may use the device 115 to establish, using a communication protocol, a multimedia session between the device 115 and a remote device 115 associated with the remote medical provider. In some examples, the device 115 may determine whether to initiate a patient transfer procedure for the patient based on the multimedia session. In some examples, the device 115 may transmit, to the remote device 115 of the remote medical provider, an indication of determining whether to initiate the patient transfer procedure for the patient. According to examples of aspects described herein, the device 115 may be associated with a personnel at a medical facility requesting the patient transfer or a remote medical provider receiving the patient transfer request. As such, the system 100 may improve efficiency, reliability and latency in healthcare environments, and more particularly for transferring patients between medical providers, thereby reducing latency involved in patient transfer procedures and ensuring that patients are provided appropriate care based on medical diagnoses.

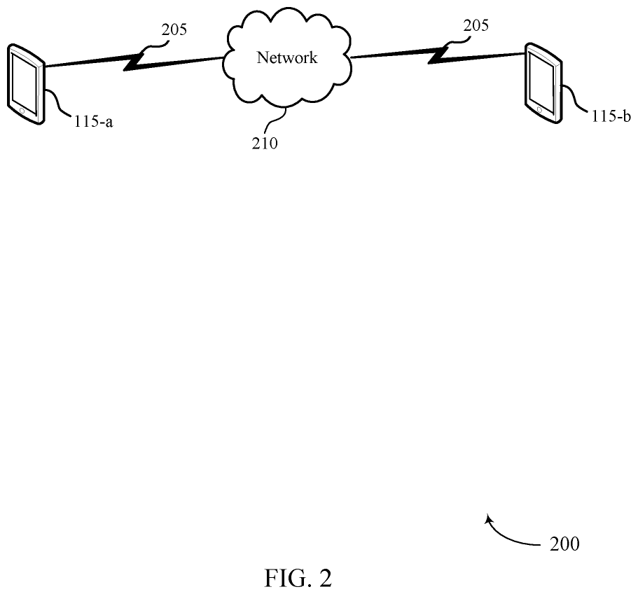

FIG. 2 illustrates an example of a system 200 that supports medical provider to medical provider communications in accordance with aspects of the present disclosure. In some examples, the system 200 may implement aspects of the system 100. For example, the system 200 may include a device 115-a and a device 115-b, which may include examples of aspects of devices 115 as described with reference to FIG. 1, to provide medical provider to medical provider communications. The device 115-a and the device 115-b may be in wireless communication via communications links 205, which may include examples of aspects of a communications link as described in FIG. 1. In some examples, the device 115-a and the device 115-b may be in wireless communication via a network 210, which may include examples of aspects of a network as described in FIG. 1. For example, the network 210 may be an LTE network, an LTE-A network, an NR network.

According to examples of aspects described herein, personnel (e.g., a physician, a hospitalist) from a medical facility may transfer patients to another medical facility through an application on the device 115-a. In an example, the device 115-a may access patient information associated with a patient via an application on the device 115-a. In an example, the device 115-a may select, via the application, a remote medical facility from a list of remote medical facilities, based on the patient information. In some aspects, the device 115-a may select a medical diagnosis for the patient. In an example, the medical diagnosis may include cardiological medical diagnosis, a neurological medical diagnosis, a trauma medical diagnosis, or a triage medical diagnosis.

The device 115-a may establish, using a communication protocol, a multimedia session between the device 115-a and a remote device 115-b associated with the remote medical facility. In some aspects, the device 115-a may determine whether to initiate a patient transfer procedure for the patient based on the multimedia session. In some example aspects, the device 115-a may transmit, to the remote device 115-b, an indication of determining whether to initiate the patient transfer procedure for the patient. According to examples of aspects described herein, the device 115-a may be associated with a user at a medical facility requesting the patient transfer, and the device 115-b may be associated with a user at a remote medical facility receiving the patient transfer request.

In an example, the device 115-a, when initiating a multimedia communication associated with transferring a patient, may select a modality associated with the treatment (e.g., cardiology, neurology, trauma, triage, psychology, social work, intensive care unit (ICU)). In some aspects, the device 115-b associated with the remote medical facility may accept, deny, or defer a request for the multimedia communication through an application on the device 115-b. In some examples, the device 115-a, may initiate a patient transfer procedure based on a user input indicative of a selection to transfer the patient to the remote medical facility. In some aspects, the device 115-b may accept or deny a request by the device 115-a associated with the transfer. Examples of aspects of establishing a multimedia communication, initiating a patient transfer procedure between medical facilities, and completing a patient transfer procedure, for example, via communications between devices 115 (e.g., the device 115-a and the device 115-b) associated with the same or different medical facilities, are described herein. In some aspects, the medical facility associated with the device 115-a establishing (e.g., initiating) the multimedia communication (and initiating the patient procedure) may be referred to, for example, as a local medical provider. The medical facility associated with the device 115-b which may accept, deny, or defer the request for the multimedia communication (and accept or deny the request associated with the transfer) may be referred to, for example, as a remote medical provider, a tertiary care center, or an urban care center. Alternatively, with respect to the perspective of the device 115-b, the medical facility associated with the device 115-a may be referred to as a remote medical provider.

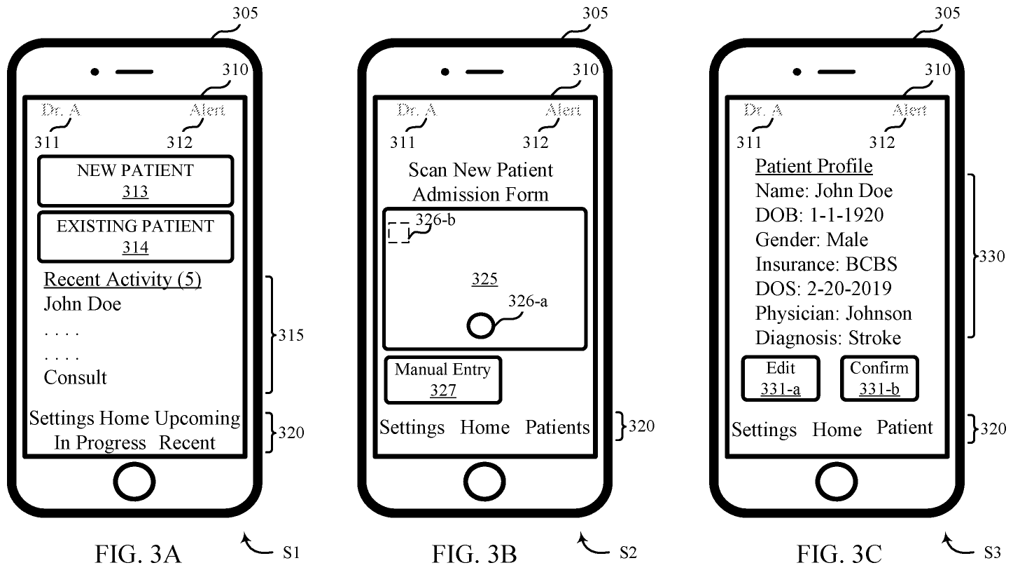

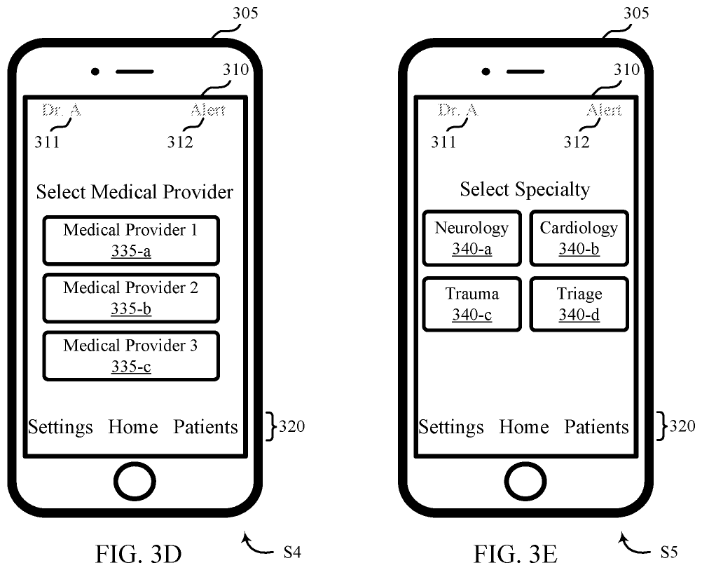

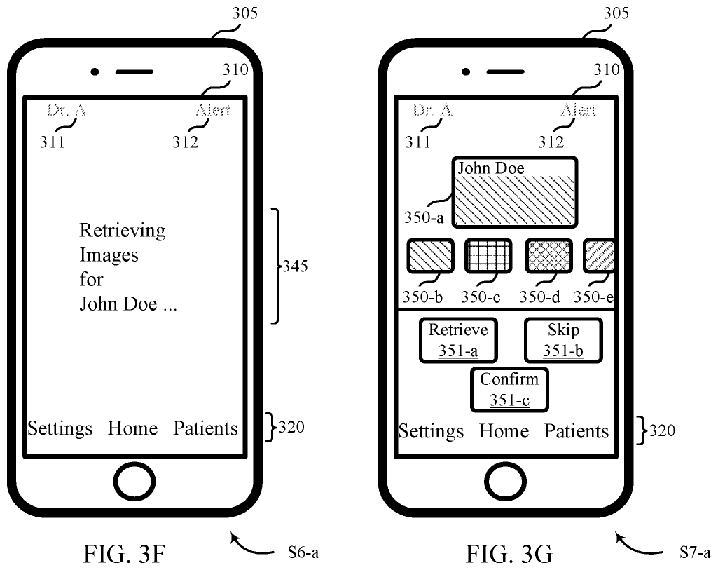

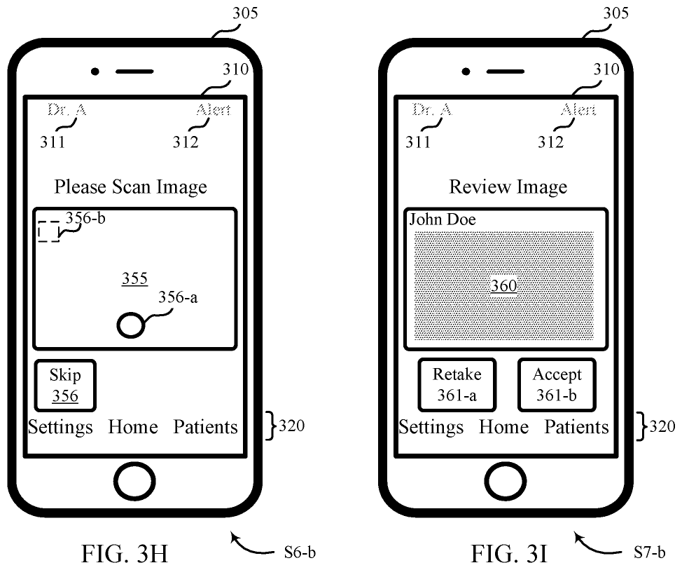

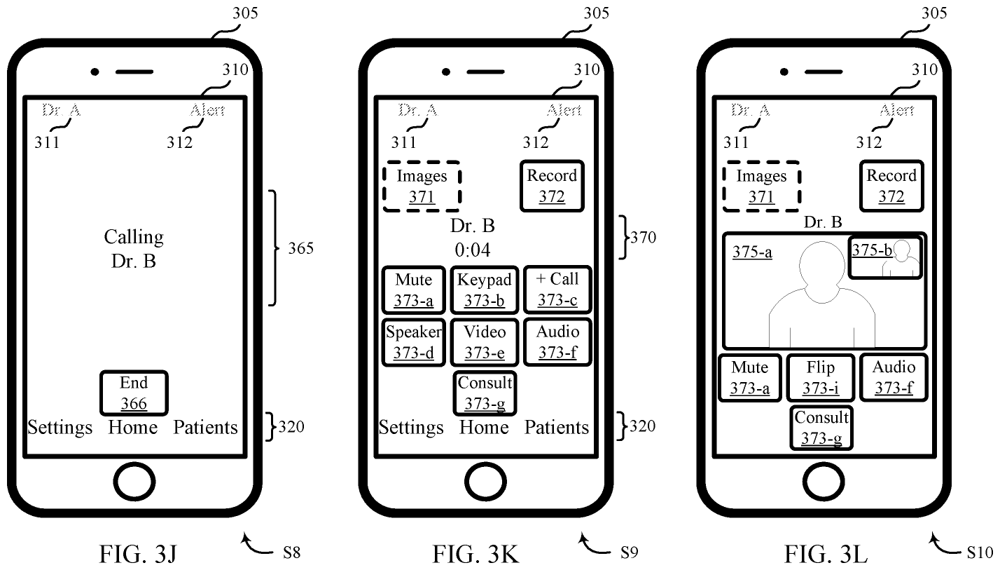

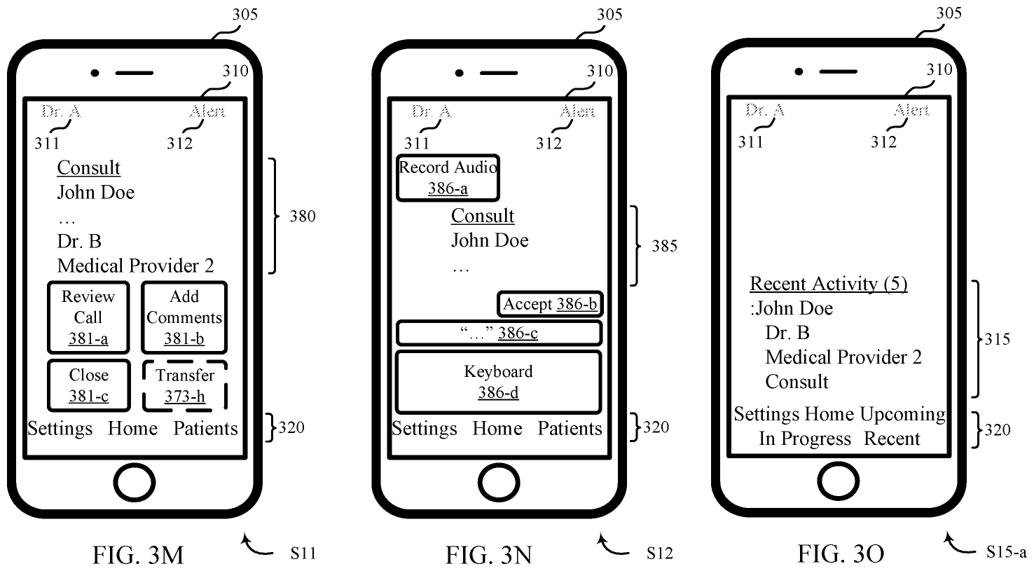

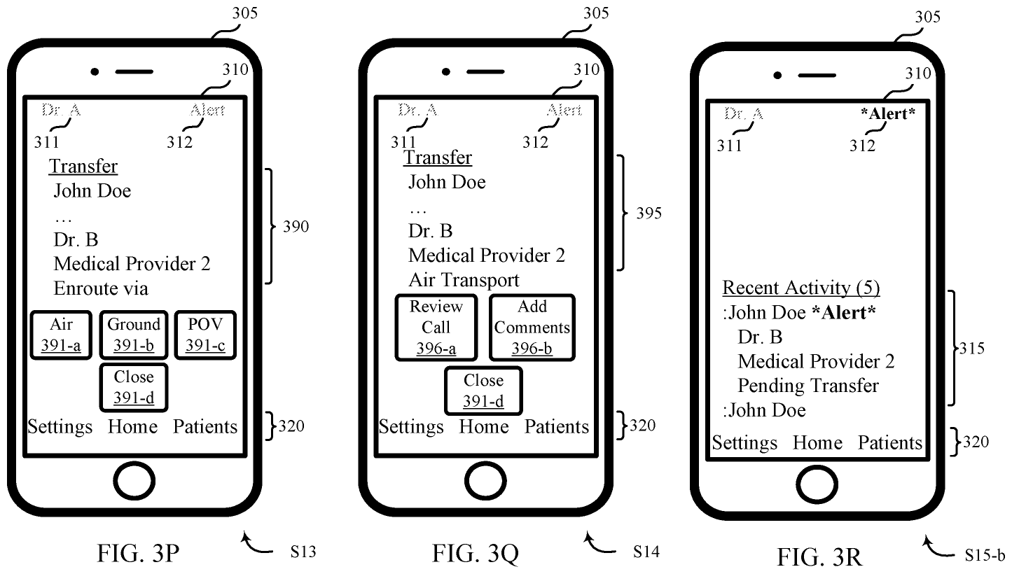

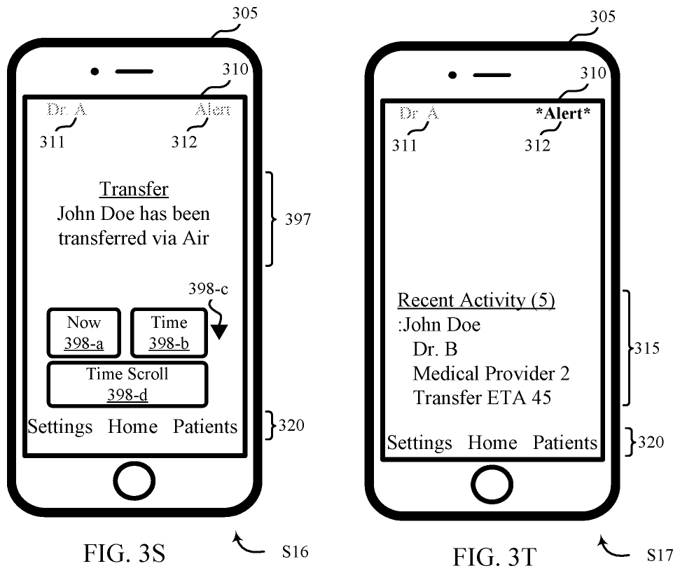

FIGS. 3A through 3T illustrate examples S1 through S17 of a device 305 that supports medical provider to medical provider communication techniques in accordance with aspects of the present disclosure. In some examples, the device 305 may implement aspects of the systems 100 and 200. For example, the device 305 may be an example of aspects of a device 115 described in FIGS. 1 and 2. The device 305 may include a user interface 310, for an example, a graphical user interface (GUI). The user interface 310 may be a display screen capable of displaying graphics information (e.g., images, video). In some examples, the user interface 310 may be a touch screen capable of recognizing user inputs (e.g., touch inputs via a user's finger, a stylus, or the like). The device 305 may also include one or more physical buttons for navigating the user interface 310.

The examples S1 through S17 illustrated in FIGS. 3A through 3T show an example process flow associated with personnel (e.g., a hospitalist, a physician, a doctor) at a medical facility (e.g., a hospital, a clinic). By way of example, the personnel may access patient information associated with a patient via an application on the device 305. The personnel may select, via the application, a remote medical facility (e.g., a hospital) or a medical diagnosis for the patient, or both, based on the patient information. In some examples, the device 305 may establish, via a communication protocol, a multimedia session between the device 305 and a remote device 305 associated with the remote medical facility. Based on the multimedia session (e.g., based on a user input associated with the multimedia session), the device 305 may determine whether to initiate a patient transfer procedure for the patient. In an example, the device 305 may transmit, to the remote device 305, an indication (e.g., a consultation indication, a transfer request) of the determination. The application on the device 305 may be an example of aspects of the application described with respect to FIGS. 1 and 2. In some aspects, following the consultation between the personnel (e.g., a treating physician) and a physician specialist at the medical facility, the application on the device 305 may provide a notification (e.g., an e-mail, a short-messing service (SMS) text message, a PDF document, etc.) to the medical facility (e.g., specialist clinic associated with the physician specialist). The notification may include patient information and information associated with the consultation, based on which personnel at the medical facility may facilitate a follow-up visit for the patient with the physician specialist.

According to examples of aspects described herein, the device 305 may execute an application supportive of medical provider to medical provider communication. The device 305 (e.g., the application) may initiate a login procedure which may prompt a user (e.g., personnel) for login credentials. In some examples, the login credentials may include user identification information such as a username, a password, a personal identification number (PIN), a fingerprint, a face scan, or the like. In some aspects, the device 305 may support the input of login credentials and data access via a server compliant with secure encryption, authentication, authorization, and data management. For example, the device 305 may support the input of login credentials and data access via a Health Insurance Portability and Accountability Act (HIPPA) compliant server.

In an example aspect, the device 305 may determine a request to access the application running on the device 305. The device 305 may provide access to the application running on the device 305 based on an authentication procedure. In some aspects, the authentication procedure may include receiving credential information via the device 305 and authenticating the device 305 based on the credential information. In some example aspects, the credential information may include a username and a password, a facial recognition, a voice recognition, a biosignature recognition, or any combination thereof.

In some other example aspects, the authentication procedure comprises a two-factor authentication procedure including receiving first credential information from via the device 305, authenticating the first credential information, receiving second credential information via the device 305 based on authenticating the first credential information, and authenticating the second credential information. In some aspects, the first credential information or the second credential information, or both may include a username and a password, a facial recognition, a voice recognition, a biosignature recognition, or any combination thereof.

With reference to S1 in FIG. 3A, the device 305 may display a main dashboard (e.g., home dashboard, home screen) on the user interface 310. For example, the device 305 may display the main dashboard based on a successful user login (e.g., user entry of valid login credentials, user verification based on valid login credentials). The main dashboard may include, for example, a profile indicator 311, an alert indicator 312, a menu 320, a “new patient” button 313, an “existing patient” button 314, a dialogue window 315, and any various additional information associated with features of the application on the device 305. In some examples, the device 305 may display the main dashboard without the “new patient” button 313 or the “existing patient” button 314. In some example aspects, the term “button” described with respect to user interfaces described herein may refer to a virtual button via which a device (e.g., device 115, device 305, device 405, device 505) may receive a user input.

The profile indicator 311 may include (e.g., display) identification information associated with the user. For example, the profile indicator 311 may include (e.g., display) the name of the user, a medical facility (e.g., a hospital, a clinic) associated with the user, a location of the medical facility, or any additional identification information associated with the user. In the example illustrated in FIG. 3A, a user “Dr. A” associated with the Medical Provider A (e.g., a medical care facility) is logged into the device 305, as shown at the profile indicator 311.

The alert indicator 312 may include (e.g., display) an alert (e.g., a notification) associated with a communication between the medical facility associated with the user and another medical facility, a consult between the medical facilities, or a patient transfer between the medical facilities. The alert indicator 312 may include text (e.g., the term “alert”), a text format (e.g., bold text, non-bold text, greyed out text), a symbol (e.g., an exclamation point, an asterisk), or a counter (e.g., number), or any combination for indicating read alerts or unread alerts. In some aspects, the device 305 may output an audible alert in combination with the displayed alert. In an example, the device 305 may output the audible alert continuously or periodically until receiving a user input associated with an unread alert (e.g., a user input selecting the unread alert). In some aspects, properties associated with the alerts (e.g., volume associated with an audio notification) may be configured based on urgency level associated with the alert.

Based on a user input associated with the alert indicator 312 (e.g., a user input selecting the alert indicator 312), the device 305 may display patient information associated with the communication, the consult, or the patient transfer, aspects of which are described herein. In some aspects, the device 305 may display the patient information without receiving the user input (e.g., via a notification message or pop-up). In the example illustrated in FIG. 3A, there are no unread alerts, and the device 305 may display “alert” greyed out in the alert indicator 312. In some aspects, the device 305 may refrain from displaying the alert indicator 312 to indicate there are no unread alerts.

The device 305 may display one or more visual elements for inputting patient information based on a user input selecting the “new patient” button 313, aspects of which are described with reference to S2 illustrated in FIG. 3B. Based on a user input selecting the “new patient” button 313, the device 305 may generate a patient account. The device 305 may store the patient account in a database remote or local to the device 305 or a server, or any combination thereof. In some aspects, the device 305 may display patient information associated with existing patients based on a user input selecting the “existing patient” button 314.

In an example of displaying patient information associated with existing patients, the device 305 may display patient information of patients seen by the personnel (e.g., patients examined by the personnel, patients examined by the user and transferred to another medical facility, or patients transferred to the personnel from another medical facility) within a time period equal to a configured duration (e.g., a 5 day period, a 15 day period, a 30 day period). The device 305 may display or filter the patient information based on, for example, date range, time (e.g., chronological order), alphabetical order, treatment category (e.g., neurology, cardiology, trauma, triage, psychology, social work, ICU). Based on user inputs selecting the “existing patient” button and selecting a patient name from a displayed list of existing patients, the device 305 may identify a patient account corresponding to a patient and access the patient account (e.g., retrieve patient information from the patient account), via the application running on the device 305 or the server, or any combination thereof.

The dialogue window 315 may include a list of activity associated with the personnel (e.g., “Dr. A”) and a set of patients seen by the personnel (e.g., patients examined or treated by the personnel, patients examined or treated by the personnel and transferred to another medical facility, or patients transferred to the personnel from another medical facility). In some aspects, the list of activity may include activity associated with a duration (e.g., activity within the last 4 hours, the last 12 hours, the last 24 hours, etc.). In some aspects, the device 305 may display or filter the list of activity based on time (e.g., chronological order), alphabetical order, treatment category or modality (e.g., neurology, cardiology, trauma, triage, psychology, social work, ICU), or any other combination of factors. In some examples, the device 305 may display or filter the list of activity based on the five most recent patients seen by the user, the five most recent patients seen by the user and transferred to another medical facility, the five most recent patients transferred to the user from another medical facility, the five most recent patients seen by the user for a neurological issue/cardiological issue/trauma issue/triage issue, etc. In an example, the device 305 may display the list of activity as “Recent Activity”, and in some aspects, display an indicator or counter (e.g., “5”) associated with the list. The dialogue window 315 may be scrollable.

In some aspects, the device 305 may provide access to patient information associated with recent activities (e.g., five patients most recently seen by the user, activity within the last 24 hours) indicated in the dialogue window 315. In an example, based on a user input selecting patient information associated with a recent activity indicated in the dialogue window 315 (e.g., a user input selecting “John Doe” from the dialogue window 315), the device 305 may retrieve images or patient information associated with John Doe. For example, the device 305 may query a picture archiving and communication system (PACS) for images (e.g., magnetic resonance imaging (MRI) brain scans) associated with John Doe, aspects of which are described with respect to FIGS. 3F and 3G. In some aspects, the device 305 may query a PACS associated with the medical facility associated with the personnel (e.g., Medical Provider A associated with Dr. A).

In another example, based on a user input selecting patient information associated with a recent activity indicated in the dialogue window 315 (e.g., a user input selecting “John Doe” from the dialogue window 315), the device 305 may display a user interface for scanning and uploading images associated with John Doe. For example, the device 305 may display a user interface for scanning and uploading cardiology picture archiving and communication systems (CPACS) images (e.g., electrocardiogram (EKG) images) associated with John Doe, aspects of which are described with respect to FIGS. 3H and 3I. In some aspects, the device 305 may display the main dashboard (e.g., home dashboard), visual elements, etc., with or without displaying the menu 320. For example, the application on the device 305 may be navigated via the menu 320 or other buttons provided by the device 305 (e.g., using physical buttons or virtual buttons of the device 305, for example, a “back” button).

In an example, at the main dashboard, the menu 320 displayed by the device 305 may include digital buttons “Settings”, “Home”, “Upcoming Calls”, “In Progress”, and “Recent Activity”. Based on a user input selecting the “Settings” button, the device 305 may display a settings menu for the application on the device 305. In some examples, based on a user input selecting the “Home” button, the device 305 may display the main dashboard. In some other examples, based on a user input selecting the “Upcoming Calls” button, the device 305 may display a list of upcoming multimedia sessions (e.g., deferred multimedia sessions). Examples of deferring multimedia sessions are described herein. In some other examples, based on a user input selecting the “In Progress” button, the device 305 may display a list of patient transfers in progress. Examples of patient transfers are described herein. In an example, based on a user input selecting the “Recent Activity” button, the device 305 may display a list of recent activity as described herein. In some example aspects, the device 305 may display any of the list of upcoming multimedia sessions, the list of patient transfers in progress, or the list of recent activity in the dialogue window 315.

FIG. 3B illustrates an example S2 where the device 305 has received a user input selecting the “new Patient” button 313. The device 305 may display one or more visual elements for inputting patient information. The visual elements may include, for example, a scanning window 325 and a “manual entry” button 327. The scanning window 325 may include a user interface for scanning a patient document (e.g., a patient admission form, a patient information form). In an example, via the scanning window 325, the device 305 may display a camera view associated with one or more sensors (e.g., cameras) coupled to the device 305. The device 305 may capture an image of the patient document, for example, based on a user input (e.g., a user input selecting a “capture” button 326-a). The device 305 may extract information from the captured image (e.g., determine information elements of the patient document) using one or more optical character recognition (OCR) operations and display the extracted information to the user, as will be described with respect to FIG. 3C. In some aspects, the device 305 may store the extracted information to memory (e.g., cache memory, random access memory (RAM)) of the device 305. In some aspects, the device 305 may store the extracted information to a database or a server (e.g., a cloud server compliant with secure encryption, authentication, authorization, and data management).

Alternatively or additionally, the device 305 may store a set of images previously captured by the device 305 (e.g., previously captured images of patient documents, stored to memory of the device 305). The device 305 may present the set of images to the user, for example, based on a user input (e.g., a user input selecting a “previously captured images” button 326-b). Based on a further user input (e.g., a user input selecting an image), for example, the device 305 may extract information from the image using one or more OCR operations and display the extracted information to the user. In some aspects, the device 305 may display one or more elements for manually entering information (e.g., patient information). For example, based on a user input selecting with the “manual entry” button 327, the device 305 may display an on-screen keyboard or user interface for manually entering the information.

FIG. 3C illustrates an example S3 where the device 305 has captured an image of the patient document, extracted information from the captured image using one or more OCR operations, and is displaying the extracted information (e.g., a “Patient Profile” page). For example, based on a user input (e.g., a user input selecting the “capture” button 326-a in FIG. 3B), the device 305 may display a patient profile inclusive of the extracted information via a dialogue window 330. The patient profile may include, for example, identification information associated with the patient (e.g., name, date of birth (DOB), gender, and insurance). In some aspects, the patient profile may include a date of service (DOS) when the patient received service (e.g., received treatment, was given an initial diagnosis) from the medical facility associated with the personnel (e.g., Medical Provider A associated with Dr. A). In some example aspects, the patient profile may include the name of the physician who provided service to the patient (e.g., Dr. Johnson), a diagnosis provided by the physician (e.g., stroke), or both.

In some aspects, the device 305 may display one or more elements for editing or confirming the patient profile. For example, the device 305 may display an “edit” button 331-a for editing the patient profile and a “confirm” button 331-b for confirming the patient profile. In some aspects, based on a user input selecting with the “edit” button 331-a, the device 305 may display an on-screen keyboard or user interface for manually entering the information. In some example aspects, based on a user input selecting with the “confirm” button 331-b, the device 305 may provide the user with options for selecting a medical facility for further diagnosis or treatment of the patient associated with the patient profile, a specialty (e.g., a medical specialty) associated with the further diagnosis or treatment, or both, as described with respect to FIG. 3D.

FIG. 3D illustrates an example S4 where the device 305 may display a list of medical facilities on the user interface 310. For example, the device 305 may display a “Medical Provider 1” button 335-a, a “Medical Provider 2” button 335-b, and a “Medical Provider 3” button 335-c. Each of the buttons 335-a through 335-c may be associated with a medical facility (e.g., “Medical Provider 1”, “Medical Provider 2”, “Medical Provider 3”) different from the medical facility associated with the personnel (e.g., different from Medical Provider A associated with Dr. A). The device 305 may initiate a multimedia session (e.g., an audio call, a video call) between the device 305 and a remote device 305 associated with any of the Medical Providers 1 through 3 based on a user input. For example, based on a user input selecting the “Medical Provider 2” button 335-b, the device 305 may initiate a multimedia session (e.g., an audio call, a video call) between the device 305 and a remote device 305 associated with the Medical Provider 2, aspects of which are described herein. Medical Providers 1 through 3 may also be referred to as remote medical facilities.

In some aspects, the device 305 may display the “Medical Provider 1” button 335-a, the “Medical Provider 2” button 335-b, and the “Medical Provider 3” button 335-c in ascending or descending order, based on geospatial information (e.g., global positioning satellite (GPS) coordinates) associated with the Medical Providers 1 through 3 and the Medical Provider A. For example, the device 305 may display the “Medical Provider 1” button 335-a, the “Medical Provider 2” button 335-b, and the “Medical Provider 3” button 335-c in ascending or descending order based on respective distances from the Medical Providers 1 through 3 to the Medical Provider A. In some example aspects, the device 305 may display the “Medical Provider 1” button 335-a, the “Medical Provider 2” button 335-b, and the “Medical Provider 3” button 335-c, for example, in ascending or descending order, based on wait times for communicating with the medical facilities, ratings, user preference (e.g., medical facilities preferred by “Dr. A”), number of available beds, or any combination thereof.

In some other example aspects, the device 305 may hide (e.g., refrain from displaying) medical facilities based on one or more criteria. For example, the device 305 may hide (e.g., refrain from displaying) any medical facilities which exceed a threshold distance from the Medical Provider A. In some examples, the device 305 may hide (e.g., refrain from displaying) any medical facilities which are outside a hospital network (e.g., Healthcare Corporation of America (HCA)) or do not accept an insurance provider (e.g., insurance coverage) associated with the patient. In an example, the device 305 may hide (e.g., refrain from displaying) any medical facilities having a wait time exceeding a threshold wait time. For example, the device 305 may hide (e.g., refrain from displaying) any medical facilities which are unavailable for communications (e.g., a multimedia session) for more than a time threshold with respect to a current temporal instance (e.g., a medical facility where staff, doctors, nurses are unavailable for a duration of 15 minutes or more from a current temporal instance, 30 minutes or more, etc.). In some aspects, the device 305 may hide (e.g., refrain from displaying) any medical facilities having no available beds for patient transfers.

Alternatively or additionally, the device 305 may differentiate the displayed medical facilities by applying different fonts, font colors, or indicators to the “Medical Provider 1” button 335-a, the “Medical Provider 2” button 335-b, and the “Medical Provider 3” button 335-c based on the criteria described herein (e.g., time threshold, threshold distance, accepted insurance, wait time threshold, number of beds threshold). For example, the device 305 may gray out a button 335 (e.g., the “Medical Provider 2” button 335-b) associated with a medical facility which exceeds a threshold distance from the Medical Provider A. In some aspects, where the device 305 grays out a button 335 (e.g., the “Medical Provider 2” button 335-b) associated with a medical facility, the device 305 may remove the option for the user to select the button 335 (e.g., the medical facility) from the list of medical facilities.

According to examples of aspects described herein, the device 305 may provide the user with options to filter or display the list of medical facilities based on the criteria described herein. For example, the device 305 may provide the user with selectable options for filtering or displaying the list of medical facilities based on criteria described herein such as distance, preferred wait times (e.g., urgent/now, 15 minutes or less, 30 minutes or less), preferred medical facilities, etc. In some example aspects, based on a user input selecting a medical facility (e.g., a user input selecting with the “Medical Provider 2” button 335-b), the device 305 may provide the user with options for selecting a modality (e.g., a specialty) for further diagnosis or treatment of the patient as described with respect to FIG. 3E.

FIG. 3E illustrates an example S5 where the device 305 may display a list of modalities (e.g., specialties) on the user interface 310. For example, the device 305 may display a “Neurology” button 340-a, a “Cardiology” button 340-b, a “Trauma” button 340-c, a “Triage” button 340-d. In some examples, the device 305 may display optional buttons including a “Call” button and an “Add Imaging” button. Each of the buttons 340-a through 340-d may be associated with a respective specialty (e.g., a specialty physician) provided by the medical facility selected by the user. For example, based on a user input selecting the “Medical Provider 2” button 335-b at FIG. 3D, the device 305 may display a “Neurology” button 340-a, a “Cardiology” button 340-b, a “Trauma” button 340-c, and a “Triage” button 340-d associated with the Medical Provider 2, as illustrated in FIG. 3E. The “Neurology” button 340-a, the “Cardiology” button 340-b, the “Trauma” button 340-c, and the “Triage” button 340-d may each be associated with a hospitalist associated with the indicated modality (e.g., specialty) at the Medical Provider 2.

In some aspects, the device 305 may display options associated with each of the modalities (e.g., specialties). For example, based on a user input selecting a modality, the device 305 may display menu (e.g., a sub-menu, a pop-up menu) associated with the selected modality. In an example, based on a user input selecting the “Neurology” button 340-a at FIG. 3E, the device 305 may display a menu including a list of subspecialties (e.g., autonomic disorders, behavioral neurology & neuropsychiatry, clinical neuromuscular pathology, geriatric neurology, etc.) associated with neurology. In some aspects, the device 305 may automatically display the menu including a list of subspecialties, for example, based on details associated with the diagnosis provided by the personnel (e.g., based on details of the diagnosis provided by Dr. A).

In some other example aspects, the device 305 may hide (e.g., refrain from displaying) modalities (e.g., specialties) from the list based on one or more criteria. For example, the device 305 may hide (e.g., refrain from displaying) any modalities associated with a wait time exceeding a threshold wait time. For example, the device 305 may hide any modalities associated with a hospitalist (e.g., a physician, a doctor) who is unavailable for communications (e.g., a multimedia session) within a time threshold (e.g., duration of 15 minutes or more from a current temporal instance, 30 minutes or more, etc.) from a current temporal instance. In some aspects, the device 305 may hide any modalities which are unavailable or not offered at the medical facility selected at FIG. 3D. In an example aspect where no neurologists are available (e.g., no neurologists are available based on time, or neurology services are not offered) at the medical facility selected in the example of FIG. 3D (e.g., the Medical Provider 2), the device 305 may hide (e.g., refrain from displaying) the “Neurology” button 340-a.

In some aspects, the device 305 may be configured based on credentials associated with the user logged into the device 305. For example, the device 305 may provide the list of modalities (e.g., specialties) based on the credentials. In an example, where the user is a cardiologist, the device 305 may hide (e.g., refrain from displaying) any modalities unrelated to cardiology. For example, the device 305 may hide (e.g., refrain from displaying the “Neurology” button 340-a, the “Trauma” button 340-c, the “Triage” button 340-d, or a combination thereof). In some example aspects, the device 305 may prioritize the display of the modalities (e.g., modalities associated with the user may be displayed by the device 305 at or towards the top of the user interface 310 illustrated in FIG. 3E).

In some aspects, the device 305 may display the modalities (e.g., specialties) different from one another based on the criteria described herein (e.g., based on hospitalist availability, based on modality available). For example, the device 305 may differentiate the modalities (e.g., specialties) by applying different fonts, font colors, or indicators to the “Neurology” button 340-a, the “Cardiology” button 340-b, the “Trauma” button 340-c, or the “Triage” button 340-d associated with the Medical Provider 2. In the example where no neurologists are available at the medical facility selected in the example of FIG. 3D (e.g., the Medical Provider 2), the device 305 may gray out the “Neurology” button 340-a. In some aspects, the device 305 may remove the option for the user to select the “Neurology” button 340-a.

In an example aspect, based on a user input selecting a modality (e.g., a user input selecting the “Neurology” button 340-a, the “Cardiology” button 340-b, the “Trauma” button 340-c, or the “Triage” button 340-d) the device 305 may provide the user with options for selecting, scanning, or uploading information (e.g., images, documents, test results) corresponding to a patient diagnosis associated with the selected modality. In an example, the device 305 may provide the user with options for selecting, scanning, or uploading information (e.g., images, documents, test results) associated with a patient diagnosis associated with a selected modality. For example, the device 305 may retrieve images corresponding to the medical diagnosis for the patient and include, in session information (e.g., a multimedia session), the images corresponding to the medical diagnosis for the patient. In some examples, based on a user input selecting a modality (e.g., a user input selecting the “Neurology” button 340-a, the “Cardiology” button 340-b, the “Trauma” button 340-c, or the “Triage” button 340-d) and the “Add Imaging” button, the device 305 may provide the user with the options for selecting, scanning, or uploading information (e.g., images, documents, test results) corresponding to a patient diagnosis associated with the selected modality. In some aspects, the device 305 may be configured to disable the “Add Imaging” button until the user has selected a modality (e.g., based on a user input selecting the “Neurology” button 340-a, the “Cardiology” button 340-b, the “Trauma” button 340-c, or the “Triage” button 340-d). In some aspects, the device 305 may be configured to enable the “Add Imaging” button regardless of whether the user has selected a modality.

In an example, based on a user input selecting the “Neurology” button 340-a, the device 305 may provide the user with options for accessing and selecting images stored on a server (e.g., PACS images, MRI brain scans) and sharing the selected images with a user at the selected medical facility (e.g., a neurologist at the selected medical facility), aspects of which are described herein with respect to the examples illustrated in FIGS. 3F and 3G. In another example, based on a user input selecting the “Cardiology” button 340-b, the device 305 may provide the user with options for scanning, uploading, and sharing images (e.g., CPACS images, EKG images) with a user at the selected medical facility (e.g., a cardiologist at the selected medical facility), aspects of which are described herein with respect to the examples illustrated in FIGS. 3H and 3I.

In the example aspects as described herein, the device 305 may display the options for selecting a medical facility, followed by displaying the options for selecting a modality. In some aspects, the device 305 may interchange the order in which the device 305 displays the options for selecting a medical facility and selecting a modality. In some alternative example aspects, the device 305 may display the options for selecting a modality, followed by displaying the options for selecting a medical facility (e.g., based on the selected medical facility). In an example, based on a user input selecting the “confirm” button 331-b as described at FIG. 3C, the device 305 may provide the user with options for selecting a modality (as illustrated in FIG. 3E). Based on a user input selecting the modality, the device 305 may display options for selecting a medical facility (as illustrated in FIG. 3D). For example, the device 305 may display a list of medical facilities which may provide services associated with the selected modality.

Based on a user input selecting a modality (e.g., the “Neurology” button 340-a), the device 305 may initiate a multimedia session (e.g., an audio call, a video call) between the device 305 and a remote device 305 associated with the selected medical facility (e.g., Medical Provider 2) and the selected modality (e.g., neurology) associated with the medical facility, aspects of which are described herein with respect to the examples illustrated in FIGS. 3J through 3L. In some examples, based on a user input selecting the modality (e.g., the “Neurology” button 340-a) and the “Call” button, the device 305 may initiate a multimedia session (e.g., an audio call, a video call) between the device 305 and a remote device 305 associated with the selected medical facility (e.g., Medical Provider 2) and the selected modality (e.g., neurology) associated with the medical facility. In an example aspect where the device 305 displays the “Call” button, the device 305 may be configured to disable the “Call” button until the user has selected a modality (e.g., based on a user input selecting the “Neurology” button 340-a, the “Cardiology” button 340-b, the “Trauma” button 340-c, or the “Triage” button 340-d). In some aspects, the device 305 may be configured to enable the “Call” button regardless of whether the user has selected a modality.

In some example aspects of initiating a multimedia session between the device 305 and a remote device 305, the device 305 may transmit, to the remote device 305 associated with the remote medical facility, a request message to establish the multimedia session. The request message may include session information for the multimedia session. The session information may include patient information or a medical diagnosis for the patient, or both. In some aspects, the device 305 may receive, from the remote device 305 associated with the remote medical facility, a response message based on the request message. The response message may include an indication of accepting, delaying, or declining the request message to establish the multimedia session. The device 305 may establishing the multimedia session between the device 305 and the remote device 305 based on the response message.

FIGS. 3F and 3G illustrate examples S6-a and S7-a where the device 305 has received a user input selecting the “Neurology” button 340-a with respect to FIG. 3E. In an example aspect, the device 305 may retrieve images (e.g., PACS images, MRI brain scans) associated with the patient John Doe from a server based on the user input. In some aspects, the device 305 may display a dialogue window 345 on the user interface 310, for example, while retrieving the images from the server. The dialogue window 345 may include, for example, a message “Retrieving images for John Doe . . . ” In some example aspects, the device 305 may display a list of images to select from among the retrieved images.

The device 305 may display the retrieved images, as shown in the example S7-a illustrated in FIG. 3G. In some aspects, the device 305 may provide a user interface via which a user may scroll (e.g., via a swipe gesture) and select between the retrieved images. With reference to S7-a in FIG. 3G, the retrieved images may include images 350-b through 350-e. In some aspects, the device may display an enlarged view or preview image (e.g., image 350-a) of a selected image. For example, based on a user input scrolling to or selecting the image 350-b, the device 305 may display the image 350-a, where the image 350-a is an enlarged view of the image 350-b.

The device 305 may display one or more elements for retrieving additional images (e.g., additional images related to the patient) or filtering the retrieved images. For example, the device 305 may display a “retrieve” button 351-a. Based on a user input selecting the “retrieve” button 351-a, the device 305 may retrieve additional images that have been uploaded to the server since images (e.g., images 350-b through 350-e) were retrieved by the device 305. In an example aspect, based on the user input selecting the “retrieve” button 351-a, the device 305 may display an interface for refining the number of images displayed (e.g., based on a temporal range). In an example, based on a user input via the interface (e.g., a user input selecting a temporal range of a 15 day period), the device 305 may retrieve or display a reduced number of images. For example, the device 305 may display the images 350-d and 350-e (e.g., where the images 350-d and 350-e were captured or have a timestamp dated within the 15 day period), but not the images 350-b and 350-c (e.g., where the images 350-d and 350-e were captured or have a timestamp dated outside the 15 day period).

The device 305 may provide an option for the user to select one or more retrieved images (e.g., one or more of the images 350-b through 350-e) and share the selected images with a user at the selected medical facility (e.g., a neurologist at the selected medical facility, a nursing supervisor at the selected medical facility). For example, the device 305 may display a “confirm” button 351-c for confirming the user selection of the retrieved images. In an example, based on a user input selecting (e.g., via a tap input, a tap and hold, a hold and drag, etc.) one or more of the retrieved images and confirming the selection via the “confirm” button 351-c, the device 305 may initiate a multimedia session (e.g., an audio call, a video call) between the device 305 and a remote device 305 associated with the user at the selected medical facility, example aspects of which are described herein with respect to the examples illustrated in FIGS. 3J through 3L. In some example aspects, based on the user input selecting one or more of the retrieved images (e.g., before confirming the selection via the “confirm” button 351-c, or after confirming the selection via the “confirm” button 351-c), the device 305 may provide an option for the user to add notes (e.g., text notes, voice notes) related to the selected images.

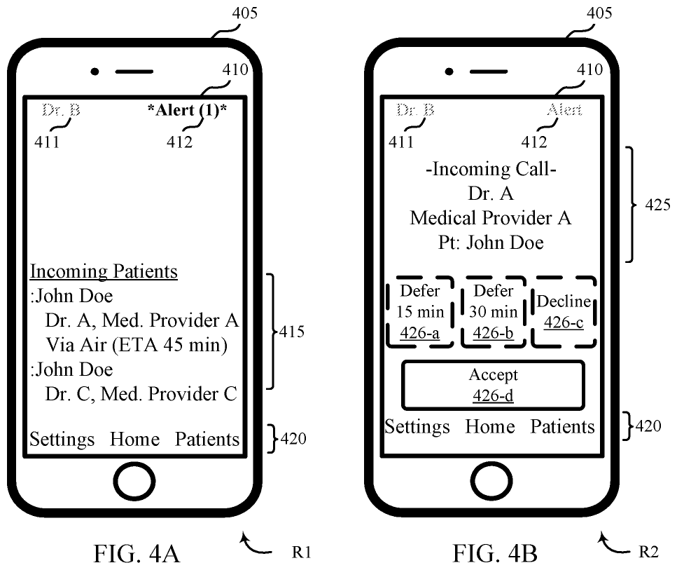

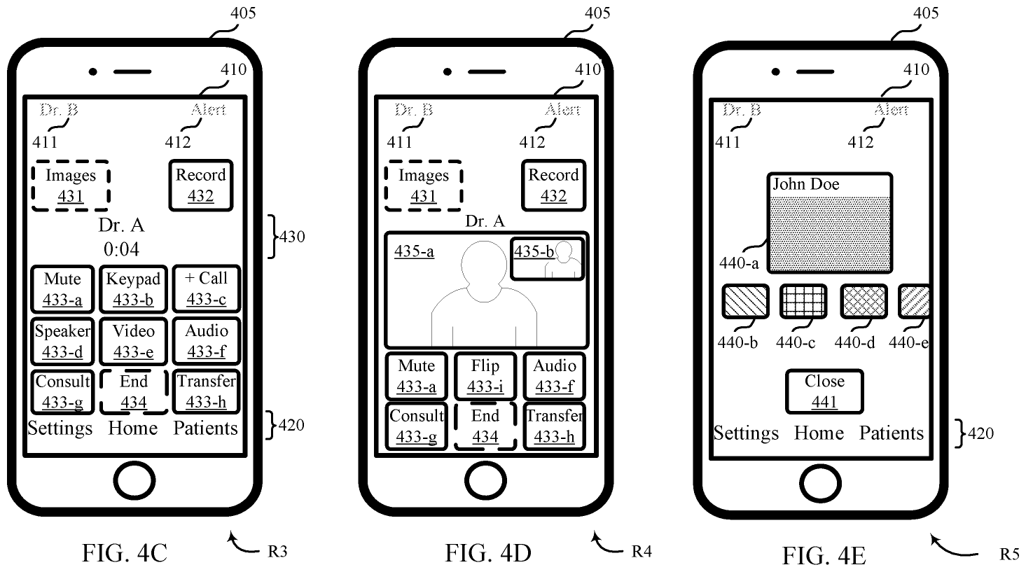

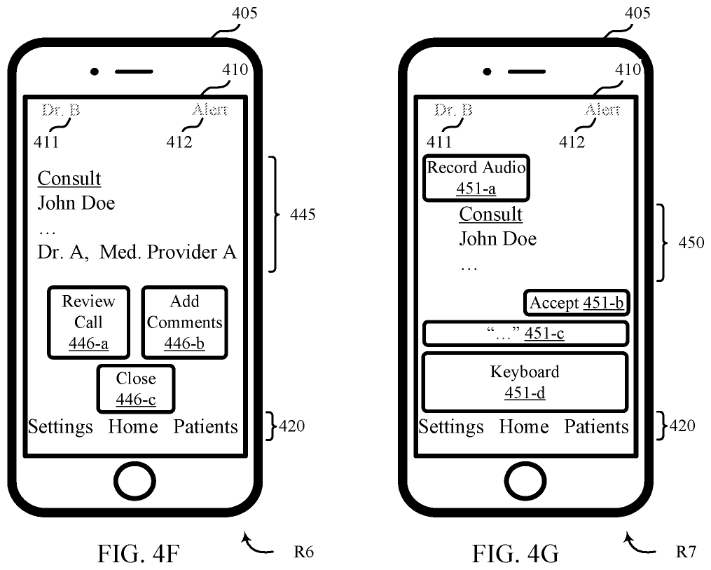

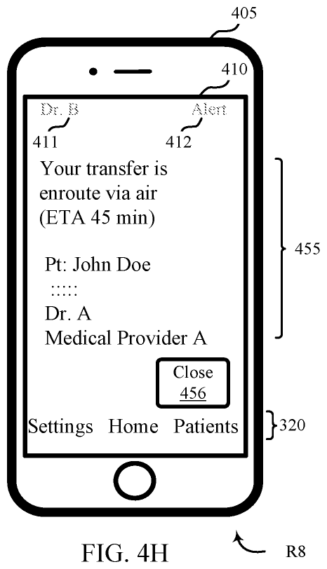

The device 305 may transmit the images to the remote device 305. In some aspects, the device 305 may transmit a link to the images (e.g., to a server hosting the images) to the remote device 305. In some other aspects, the device 305 may provide (e.g., transmit) an authorization to the remote device 305 to access the images via the server, and the remote device 305 may display an option for the user at the remote device 305 to view the images. In some aspects, the device 305 may load or upload images to the server. Example aspects of the multimedia session and sharing the selected images are described herein with respect to FIGS. 3J through 3L and FIGS. 4C through 4E. In some example aspects, the device 305 may provide an option for the user to add notes (e.g., via a user interface, for example, an on-screen keyboard) related to the selected images, append and share additional documents (e.g., examination reports, medical reports) related to the selected images, or both.

In some aspects, the device 305 may display one or more elements for skipping the option for sharing retrieved images. For example, the device 305 may display a “skip” button 351-b. In an example, based on a user input selecting the “skip” button 351-b and confirming the selection via the “confirm” button 351-c, the device 305 may initiate a multimedia session (e.g., an audio call, a video call) between the device 305 and a remote device 305 associated with the user at the selected medical facility, without sharing one or more of the images with the remote device 305 (e.g., without transmitting the images, without transmitting the link to the images, without providing the authorization for the remote device 305 to access the images via the server). In an example, no retrieved images may be available or none of the retrieved images may be related to an illness associated with the selected modality (e.g., neurology). Based on a user input selecting the “skip” button 351-b and confirming the selection via the “confirm” button 351-c, the device 305 may initiate a multimedia session (e.g., an audio call, a video call) between the device 305 and a remote device 305 associated with the user at the selected medical facility, without sharing any images with the remote device 305. In some example aspects, based on a user input selecting the “skip” button 351-b (e.g., without a user input selecting the “confirm” button 351-c), the device 305 may initiate a multimedia session.

FIGS. 3H and 3I illustrate examples S6-b and S7-b where the device 305 has received a user input selecting the “Cardiology” button 340-b and the “Add Imaging” button with respect to FIG. 3E. In an example aspect, the device 305 may display one or more visual elements for inputting patient information associated with the modality cardiology. The visual elements may include, for example, a scanning window 355 and a “skip” button 356, as shown in the example S6-b illustrated in FIG. 3H. The scanning window 355 may include a user interface for scanning in a patient image (e.g., CPACS images, EKG images) or patient document (e.g., a medical record, a test result). In an example, via the scanning window 355, the device 305 may display a camera view associated with one or more sensors (e.g., cameras) coupled to the device 305. The scanning window 355 may include indicators for identifying edges of the patient image (or patient document) to be scanned. In some aspects, the device 305 may utilize OCR scanning techniques for identifying the edges. The device 305 may capture (e.g., scan) an image of the patient image, for example, based on a user input (e.g., a user input selecting a “capture” button 356-a).

Alternatively or additionally, the device 305 may store a set of images previously captured by the device 305 (e.g., previous captures, that is, scans of CPACS images, EKG images, stored to memory of the device 305). The device 305 may store the set of images, for example, to a memory of the device 305, cloud server (e.g., a cloud server compliant with secure encryption, authentication, authorization, and data management), or both. The device 305 may present the set of images to the user, for example, based on a user input (e.g., a user input selecting a “previously captured images” button 356-b).

The device 305 may provide an option for the user to review the captured (e.g., scanned) image and share the captured images with a user at the selected medical facility (e.g., a cardiologist at the selected medical facility, a nursing supervisor at the selected medical facility). For example, as shown in the example S7-b illustrated in FIG. 3I, the device 305 may display a preview window 360, a “retake” button 361-a, and an “accept” button 361-b for confirming the captured (e.g., scanned) image displayed in the preview window 360. In an example, based on a user input selecting the “accept” button 361-b, the device 305 may initiate a multimedia session (e.g., an audio call, a video call) between the device 305 and a remote device 305 associated with the user at the selected medical facility, example aspects of which are described herein with respect to the examples illustrated in FIGS. 3J through 3L. In some aspects, based on the user input selecting the “accept” button 361-b, the device 305 may provide an option to capture (e.g., scan) additional images prior to initiating a multimedia session.

The device 305 may transmit the captured (e.g., scanned) images to the remote device 305. In some aspects, the device 305 may upload the images to a server and transmit a link to the images (e.g., to a server hosting the images) to the remote device 305. In some other aspects, the device 305 may provide (e.g., transmit) an authorization to the remote device 305 to access the images via the server, and the remote device 305 may display an option for the user at the remote device 305 to view the images. In some example aspects, the device 305 may provide an option for the user to add notes (e.g., via a user interface, for example, an on-screen keyboard) related to the captured (e.g., scanned) images, append and share additional documents (e.g., examination reports, medical reports) related to the selected images, or both. In some additional aspects, the device 105 may display a notification indicating percentage complete associated with uploading the captured (e.g., scanned) images.

In some aspects, the device 305 may display one or more elements for skipping the option for scanning in a patient image. For example, referring to the example S6-b illustrated in FIG. 3H, the device 305 may display a “skip” button 356. In an example, based on a user input selecting the “skip” button 356, the device 305 may initiate a multimedia session (e.g., an audio call, a video call) between the device 305 and a remote device 305 associated with the user at the selected medical facility, without sharing any images with the remote device 305 (e.g., without transmitting any captured images).

FIGS. 3J through 3L illustrate examples S8 through S10 of a multimedia session between the device 305 and a remote device 305. In some aspects, FIGS. 3J through 3L may illustrate examples S8 through S10 where the device 305 has received a user input selecting the “Call” button with respect to FIG. 3E (e.g., “Neurology” modality). In some aspects, FIGS. 3J through 3L may illustrate examples S8 through S10 where the device 305 has received a user input selecting the “Accept” button 361-b with respect to FIG. 3I (e.g., “Cardiology” modality). In an example aspect, the device 305 may initiate a multimedia session between the device 305 (e.g., associated with a user, Dr. A) and the remote device 305 at the selected medical facility (e.g., associated with a user, Dr. B) based on the user input. Initiating the multimedia session may include, for example, initiating a call request (e.g., a request for an audio call or a video call).

With reference to S8 in FIG. 3J, the device 305 may display a dialogue window 365 and an “End” button 366 on the user interface 310, for example, while initiating the multimedia session or waiting for the remote device 305 to join the multimedia session (e.g., while waiting for Dr. B to join the multimedia session). In some example aspects, the device 305 may continue to display the “End” button 366 on the user interface 310 during the multimedia session (e.g., through FIGS. 3K and 3L). In some example aspects, the device 305 may hide the “End” button 366 once the multimedia session has been established (e.g., the remote device 305 has joined the multimedia session). The dialogue window 365 may include, for example, a message “Calling Dr. B.” The “End” button 366 may provide functionality for ending an initiated call request. With reference to S8 in FIG. 3J, based on a user input selecting the “End” button 366, the device 305 may cancel the initiated call request. The “End” button 366 may provide functionality for ending a multimedia session (e.g., a video call, an audio call). With reference to S9 and S10 in FIGS. 3K and 3L, based on a user input selecting the “End” button 366, the device 305 may end the multimedia session (e.g., without selecting the “Consult” button 373-g). In some example aspects, based on the user input selecting the “End” button 366, the device 305 may return the user to the main dashboard (e.g., FIG. 3A) or the “Patient Profile” page (e.g., FIG. 3C). In some example aspects, based on the user input selecting the “End” button 366, the device 305 may return the user to the list of medical facilities (e.g., FIG. 3D) or the list of modalities (e.g., FIG. 3E).

In some example aspects, the device 305 display multiple visual elements during a multimedia session. In the examples S9 and S10 illustrated in FIGS. 3K and 3L, the visual elements may include, for example, a dialogue window 370, an “Images” button 371, a “Record” button 372, a “Mute” button 373-a, a “Keypad” button 373-b, a “+ Call” button 373-c, a “Speaker” button 373-d, a “Video” button 373-e, an “Audio” button 373-f, a “Consult” button 373-g, a “Flip” button 373-I, a window 375-a, and a preview window 375-b. In some examples, the device 305 may display a “Transfer” button 373-h. In some aspects of the examples S9 and S10, the device 305 may omit the “Transfer” button 373-h. The dialogue window 370 may include a timer indicating a duration of the multimedia session. In some aspects, the dialogue window 370 may include the name of the personnel (e.g., Dr. B) associated with the remote device 305.

The “Images” button 371 may provide functionality for transmitting or sharing one or more images to the remote device 305. The device 305 may transmit or share one or more images to the remote device 305 based on a user input selecting the “Images” button 371. For example, the device 305 may transmit or share one or more of the images retrieved and selected in the examples S6-a and S7-a described with respect to FIGS. 3F and 3G (e.g., “Neurology” modality). In another example, based on a user input selecting the “Images” button 371, the device 305 may transmit or share one or more of the images captured (e.g., scanned) and uploaded in the examples S6-b and S7-b described with respect to FIGS. 3H and 3I (e.g., “Cardiology” modality).

The device 305 may transmit selected images to the remote device 305, transmit a link to the images (e.g., to a server hosting the images) to the remote device 305, provide (e.g., transmit) an authorization to the remote device 305 to access the images via the server, or a combination thereof. In some example aspects, the remote device 305 may display an option for the user at the remote device 305 to view the images, example aspects of which will be described with respect to FIGS. 4C through 4E. The “Record” button 372 may provide functionality for recording the multimedia session. For example, based on a user input selecting the “Record” button 372, the device 305 may record audio, video, or both associated with the multimedia session. In some examples, a user may select the option to record the multimedia session based on user consent. In some aspects, the device 305 may automatically initiate recording the audio, video, or both associated with the multimedia session when initiating the multimedia session or once the multimedia session has been established (e.g., based on a user input accepting the request for the multimedia session). In some example aspects, the device 305 may display an indicator (e.g., an icon), highlight the “Record” button 372, display a timer, or any combination to indicate the device 305 is recording the multimedia communication.

The “Mute” button 373-a may provide functionality for muting audio associated with the multimedia communication. For example, based on a user input selecting the “Mute” button 373-a, the device 305 may mute a microphone of the device 305 during the multimedia communication. In some aspects, the device 305 may mute the microphone of the device 305 during an audio communication or video communication. The “Keypad” button 373-b may provide functionality for entering information (e.g., numbers, text) with respect to a field displayed on the user interface 310. For example, based on a user input selecting the “Keypad” button 373-b, the device 305 may display a keypad (e.g., a number pad, a keyboard) on the user interface 310. The “+ Call” button 373-c may provide functionality for adding another device 305 (e.g., another user associated with another device 305) to the multimedia communication. For example, based on a user input selecting the “+ Call” button 373-c, the device 305 may display a keypad or interface (e.g., a dialer screen, a contacts list) on the user interface 310.

The “Speaker” button 373-d may provide functionality for toggling a speakerphone mode of the device 305 on or off. For example, based on a user input selecting the “Speaker” button 373-d, the device 305 may switch between outputting audio of the multimedia communication via an earpiece of the device 305 and a speaker of the device 305. The “Video” button 373-e and “Audio” button 373-f may provide functionality for toggling video streaming on and off during the multimedia communication. For example, based on a user input selecting the “Video” button 373-e, the device 305 may enable video streaming and audio streaming for the multimedia session, example aspects of which are described with respect to FIG. 3L. The “Flip” button 373-i may provide functionality for switching between sensors of the device 305 (e.g., switching between a front facing camera of the device 305, a rear facing camera of the device 305, or any camera electrically connected to the device 305). In another example, based on a user input selecting the “Audio” button 373-f, the device 305 may enable audio streaming (e.g., without video streaming) for the multimedia session.

The device 305 may display the window 375-a during the multimedia session. In an example where video streaming is enabled for the multimedia session, the window 375-a may include a video image received by the device 305 from the remote device 305 (e.g., a video stream from the remote device 305). In an example where audio streaming is enabled (e.g., without video streaming) for the multimedia session, the window 375-a may include a static image, for example, a logo (e.g., a logo associated with the application, a logo associated with the other medical facility). The device 305 may display the preview window 375-b during the multimedia session. In an example, the preview window 375-b may include images or documents shared from the device 305 to the remote device 305. The device 305 may display the preview window 375-b on the user interface 310. In the example where video streaming is enabled for the multimedia session, the preview window 375-b may include video images captured by the device 305 and transmitted to the remote device 305 (e.g., video streaming), images or documents captured by the device 305 and shared with the remote device 305 (e.g., PACS images, CPACS images, or scanned documents, shared via transmission from the device 305 or sharing via a server), or both.

The “Consult” button 373-g may provide functionality for ending a multimedia session (e.g., a video call, an audio call), reviewing the multimedia session, inputting comments regarding the multimedia session, initiating a patient transfer, or any combination thereof. For example, based on a user input selecting the “Consult” button 373-g, the device 305 may end the multimedia session and display one or more visual elements for reviewing the multimedia session or inputting comments regarding the multimedia session. The “Transfer” button 373-h may provide functionality for initiating a patient transfer to another medical facility. For example, based on a user input selecting the “Transfer” button 373-h, the device 305 may initiate a patient transfer to the selected medical facility (e.g., the Medical Provider B). In some examples, the device 305 may display the “Transfer” button 373-h based on a user input selecting the “Consult” button 373-g, aspects of which are described with reference to FIG. 3M. In some aspects, the device 305 may display one or more visual elements for reviewing the multimedia session, inputting comments regarding the multimedia session, and requesting the patient transfer.

In an example where the multimedia session is being recorded (e.g., the device 305 has automatically initiated the recording, or the user has selected the “Record” button 372) and the user unselects the “Record” button 372 (e.g., stops the recording), the device 305 may store a recording of the multimedia session (e.g., audio, video, or both) to a patient profile (e.g., a patient profile stored on memory of the device 305, a cloud server, or both). In another example, where the multimedia session is being recorded (e.g., the device 305 has automatically initiated the recording, or the user has selected the “Record” button 372) and the user selects the “End” button 366, the “Consult” button 373-g, or the “Transfer” button 373-h, the device 305 may store a recording of the multimedia session to the patient profile (e.g., the patient profile stored on a memory of the device 305, a cloud server, or both). In some aspects, the device 305 may generate a transcription of the recording of the multimedia session (e.g., generate a transcription of an audio portion of the recording). The device 305 may store the transcription to a memory of the device 305, a cloud server, or both. In an example, the device 305 may transmit the transcription to the remote device 305. In some example aspects, the device 305 may transmit the recording of the multimedia session to a cloud server, a third party, or both. The cloud server or the third party may generate a transcription of the recording of the multimedia session. In an example, the device 305, the remote device 305, or both may receive the transcription from the cloud server or the third party.

FIGS. 3M through 3O illustrate examples S11, S12, and S15-a where the device 305 has received a user input selecting the “Consult” button 373-g with respect to any of FIGS. 3K through 3L. With reference to S11 in FIG. 3M, the device 305 may display a dialogue window 380, a “Review Call” button 381-a, an “Add Comments” button 381-b, and a “Close” button 381-c based on the user input. In some aspects, the device 305 may also display the “Transfer” button 373-h. The dialogue window 380 may include, for example, a message including details regarding the multimedia communication. For example, the message may be a “Consult” message including the name of the patient, a diagnosis associated with the patient, users associated with the multimedia communication (e.g., Dr. A, Dr. B), medical facilities associated with the users (e.g., Medical Provider A, Medical Provider 2), or any combination thereof. In an example, the dialogue window 380 may include a message “Consult. John Doe . . . Dr. B. Medical Provider 2.”

The “Review Call” button 381-a may provide functionality for reviewing audio associated with the multimedia session, video associated with the multimedia session, a transcription associated with the multimedia session, or any combination. For example, based on a user input selecting the “Review Call” button 381-a, the device 305 may output a user interface for accessing the recording of the multimedia session (e.g., playing recorded audio or video), accessing the transcription of the recording (e.g., reading or editing transcribed audio), or both. In some aspects, the device 305 may gray out the “Review Call” button 381-a until the device 305 has processed the recording (e.g., stored the recording, uploaded the recording, transcribed the audio of the recording). In some aspects, the device 305 may gray out the “Review Call” button 381-a until the server (e.g., cloud server) has processed the recording (e.g., stored the recording, uploaded the recording, transcribed the audio of the recording). The “Add Comments” button 381-b may provide functionality for inputting comments regarding the multimedia session. For example, based on a user input selecting the “Add Comments” button 381-b, the device 305 may display visual elements for displaying and inputting comments regarding the multimedia session (e.g., the device 305 may display a comments screen). Aspects of the visual elements for displaying and inputting comments are described with respect to example S12 illustrated in FIG. 3N.

In some aspects, based on the user input selecting the “Close” button 381-c, the device 305 may return the user to the main dashboard, as shown in example S15-a illustrated in FIG. 3O. In some examples, the device 305 may return the user to the main dashboard, with or without displaying the “new patient” button 313 or the “existing patient” button 314. In some example aspects, based on the user input selecting the “Close” button 381-c, the device 305 may display a prompt for initiating or not initiating a patient transfer process. At the prompt, based on a user input selecting to initiate the patient transfer process, the device 305 may initiate the patient transfer process, aspects of which are described with respect to the examples S13 and S14 illustrated in FIGS. 3P and 3Q. In some examples, based on a user input selecting the “Transfer” button 373-h, the device 305 may initiate the patient transfer process.

With reference to S12 in FIG. 3N, the device 305 may display a dialogue window 385, a “Record Audio” button 386-a, an “Accept” button 386-b, a text window 386-c, and a keyboard 386-d. The dialogue window 385 may include, for example, information regarding the multimedia communication. In some aspects, the dialogue window 385 may include a “Consult” message including the name of the patient, the diagnosis associated with the patient, users associated with the multimedia communication, and the medical facilities associated with the users. Based on a user input, the device 305 may edit or confirm (e.g., accept) the information included in the dialogue window 385. In some aspects, the device 305 may provide message board functionality. For example, the dialogue window 385 may include a message board via which the device 305 may display comments input by participants of the multimedia communication (e.g., “Dr. A” and “Dr. B”) via the device 305 or the remote device 305. In an example, the device 305 may display the comments as a discussion thread.

The device 305 may provide functionality for a user to add, edit, or delete comments from the message board. In some aspects, the device 305 or an application (on the device 305) may encrypt comments input by a user and upload the encrypted comments to the message board (e.g., to a server hosting the message board). In some example aspects, the device 305 or the server may maintain a comments history of when a comment was added, edited, or deleted. The server may utilize encryption techniques for protecting the comments history, user information associated with the comments, device information associated with users who added the comments, etc. In some aspects, the server may communicate the comments history to the device 305 (e.g., via encrypted communications). In an example, the device 305 may display indicators (e.g., timestamps, preconfigured text such as “comment deleted”) conveying the additions, edits, or deletions.

The “Record Audio” button 386-a may provide functionality for inputting audio notes (e.g., voice notes) regarding the multimedia session. For example, based on a user input selecting the “Record Audio” button 386-a, the device 305 may record audio notes (e.g., voice notes) input by the user. In some aspects, the device 305 may display an indicator (e.g., an icon), highlight the “Record Audio” button 386-a, display a timer, or any combination to indicate the device 305 is recording the multimedia communication. In an example where the user has selected the “Record Audio” button 386-a and again selects the “Record Audio” button 386-a (e.g., stops the recording), the device 305 may store a recording of the audio notes to a memory of the device 305, a cloud server, or both.

In some aspects, the device 305 or the server may provide (e.g., grant) access to the audio notes to the user of the device 305 (e.g., Dr. A). In an example, the device 305 may provide a user interface for the user to review (e.g., play), modify, or delete the recording. In some example aspects, the device 305 or the server may provide (e.g., grant) access to the audio notes to users other than the user of the device 305. For example, based on a user input from the user of the device 305 (e.g., a permission setting by Dr. A), the device 305, the cloud server, or both may provide (e.g., grant) access to the audio notes to the user of the remote device 305 (e.g., Dr. B, a nursing supervisor, authorized medical staff). In some aspects, the device 305 may generate a transcription of the audio notes. The device 305 may store the transcription to a patient profile (e.g., a patient profile stored on a memory of the device 305, a cloud server, or both). In some aspects, the device 305 may append the transcription (or a link to the cloud server for accessing the transcription) to the dialogue window 385. In some aspects, the device 305 may output the transcription to the text window 386-c.

In some example aspects, the device 305 may transmit the recording of the multimedia session to a cloud server, a third party, or both. The cloud server or the third party may generate a transcription of the recording of the multimedia session. In an example, the device 305, the remote device 305, or both may receive the transcription from the cloud server or the third party. In some aspects, the device 305 or the cloud server may provide (e.g., grant) access to the transcription to the user of the device 305 (e.g., Dr. A). In an example, the device 305 may provide a user interface for the user to review, modify, or delete the transcription. In some example aspects, the device 305 or the server may provide (e.g., grant) access to the transcription to users other than the user of the device 305. For example, based on a user input from the user of the device 305 (e.g., a permission setting by Dr. A), the device 305, the cloud server, or both may provide (e.g., grant) access to the transcription to the user of the remote device 305 (e.g., Dr. B).

The “Accept” button 386-b may provide functionality for accepting text displayed in the dialogue window 385, the text window 386-c, or both. In an example, based on a user input selecting the “Accept” button 386-b, the device 305 may append text included in the text window 386-c to the information included in the dialogue window 385. In an example, based on the user input, the device 305 may confirm (e.g., save) the information included in the dialogue window 385. In some aspects, based on the user input selecting the “Accept” button 386-b, the device 305 may return the user to the user interface 310 shown in the example S11 illustrated in FIG. 3M. In some aspects, based on the user input selecting the “Accept” button 386-b, the device 305 may return the user to the main dashboard, as shown in example S15-a illustrated in FIG. 3O. In some examples, the device 305 may return the user to the main dashboard, with or without displaying the “new patient” button 313 or the “existing patient” button 314.

The keyboard 386-d may provide functionality for entering or editing text included in the dialogue window 385, the text window 386-c, or both. In some aspects, the dialogue window 380, the dialogue window 385, or both may include a summary (e.g., a call summary) regarding the multimedia communication. The summary may include a condensed version of the information described herein regarding the multimedia communication. In an example aspect, the device 305 may display all the information, a portion of the information, or a condensed version of the information. In some aspects, the device 305 may add all comments or the summary (e.g., the call summary) regarding the multimedia communication to a patient profile. In some example aspects, based on the user input selecting the “Close” button 381-c, the device 305 may return the user to the main dashboard, as shown in example S15-a illustrated in FIG. 3O. In an example, the device 305 may update the dialogue window 315 with information (e.g., “John Doe, Dr. B, Medical Provider 2, Consult”) associated with the multimedia session.

FIGS. 3P and 3Q illustrate examples S13 and S14 where the device 305 has received a user input selecting the “Transfer” button 373-h with respect to any of FIGS. 3K through 3M. With reference to S13 in FIG. 3P, the device 305 may display a dialogue window 390, an “Air” button 391-a, a “Ground” button 391-b, a “POV” button 391-c, and a “Close” button 391-d based on the user input selecting the “Transfer” button 373-h with respect to any of FIGS. 3K through 3M. In some aspects, the device 305 may display the dialogue window 390, the “Air” button 391-a, the “Ground” button 391-b, the “POV” button 391-c, and the “Close” button 391-d based on a user input via the dialogue window 315 (e.g., a user input selecting an activity from the list of “Recent Activity”) described with respect to any of FIGS. 3A, 3O, 3R, and 3T.

The dialogue window 390 may include, for example, a message including details regarding transferring the patient from the medical facility associated with the device 305 (e.g., from the Medical Provider A) to the selected medical facility associated with the remote device 305 (e.g., to the Medical Provider 2). For example, the message may be a “Transfer” message including the name of the patient, a diagnosis associated with the patient, users (e.g., Dr. A, Dr. B) associated with the multimedia communication or the transfer, medical facilities (e.g., Medical Provider A, Medical Provider 2) associated with the users, an indication that the patient is enroute, or any combination thereof. In an example, the dialogue window 390 may include a message “Transfer. John Doe . . . Dr. B. Medical Provider 2. Enroute via.”

The “Air” button 391-a, the “Ground” button 391-b, and the “POV” button 391-c may provide functionality for indicating a transportation type via which the patient will be transferred (e.g., transported) from the medical facility to the selected medical facility. In some aspects, the device 305 may gray out any of the “Air” button 391-a, the “Ground” button 391-b, and the “POV” button 391-c based on vehicle availability. In an example where a helicopter is unavailable, the device 305 may gray out the “Air” button 391-a and disable the option for a user input to select the “Air” button 391-a. In some additional aspects, the device 305 may gray out any of the “Air” button 391-a, the “Ground” button 391-b, and the “POV” button 391-c based on an urgency level, a condition of the patient, a time threshold, or any combination. In an example case where the diagnosis or transfer request indicates a recommendation for immediate treatment for the patient at the selected medical facility (e.g., treatment within 1 hour at the “Medical Provider B”), the device 305 may gray out the “Ground” button 391-b and the “POV” button 391-c and disable the option for the user input to select the “Ground” button 391-b or the “POV” button 391-c.

With reference to S14 in FIG. 3Q, the device 305 may display a dialogue window 395, a “Review Call” button 396-a, an “Add Comments” button 396-b, and a “Close” button 381-c based on a user input selecting the “Air” button 391-a, the “Ground” button 391-b, or the “POV” button 391-c described with respect to FIG. 3P. In some other aspects, the device 305 may display the dialogue window 395, the “Review Call” button 396-a, the “Add Comments” button 396-b, and the “Close” button 391-d based on a user input via the dialogue window 315 (e.g., a user input selecting an activity from the list of “Recent Activity”) described with respect to any of FIGS. 3A, 3O, 3R, and 3T. The dialogue window 395 may include, for example, a message including details regarding transferring the patient from the medical facility associated with the device 305 (e.g., from the Medical Provider A) to the selected medical facility associated with the remote device 305 (e.g., to the Medical Provider 2). For example, the message may be a “Transfer” message including the name of the patient, a diagnosis associated with the patient, users associated with the multimedia communication (e.g., Dr. A, Dr. B) or the transfer, medical facilities associated with the users (e.g., Medical Provider A, Medical Provider 2), an indication of the type of transportation, or any combination thereof. In an example, the dialogue window 390 may include a message “Transfer. John Doe . . . Dr. B. Medical Provider 2. Air Transport.”

The “Review Call” button 396-a may provide functionality for reviewing audio associated with the multimedia session, video associated with the multimedia session, a transcription associated with the multimedia session, or any combination. The “Review Call” button 396-a and functionalities associated therewith may include examples of aspects of the “Review Call” button 381-a described herein. The “Add Comments” button 396-b may provide functionality for inputting comments regarding the multimedia session. The “Add Comments” button 396-b and functionalities associated therewith may include examples of aspects of the “Add Comments” button 381-b described herein.

In some aspects, based on the user input selecting the “Close” button 391-d with respect to any of FIGS. 3P and 3Q, the device 305 may complete the transfer request. For example, the device 305 may submit the transfer request to the selected medical facility (e.g., the Medical Provider B). In some aspects, the device 305 may submit the transfer request to the remote device 305 associated with the selected medical facility, for example, via wireless communications system 100 or 200, a cloud server, or both. Based on a user input from personnel (e.g., Dr. B, a nursing supervisor) at the selected medical facility (e.g., via a remote device 305 associated with the selected medical facility), the device 305 may receive a response associated with the transfer request (e.g., a response accepting, denying, or delaying the transfer). According to examples of aspects described herein, the selected medical facility may be referred to as the “remote medical facility.”

In an example aspect, based on the user input selecting the “Close” button 391-d with respect to any of FIGS. 3P and 3Q, the device 305 may return the user to the main dashboard, as shown in example S15-b illustrated in FIG. 3R. In an example, the device 305 may update the dialogue window 315 with information (e.g., “John Doe, Dr. B, Medical Provider 2, Pending Transfer”) associated with the transfer request. In some examples, the device 305 may return the user to the main dashboard, with or without displaying the “new patient” button 313 or the “existing patient” button 314.

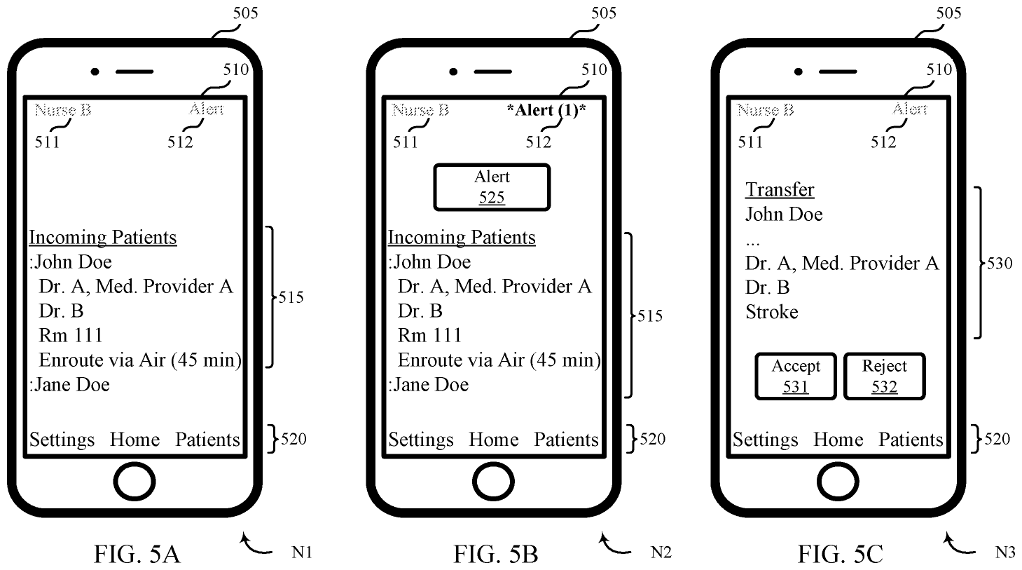

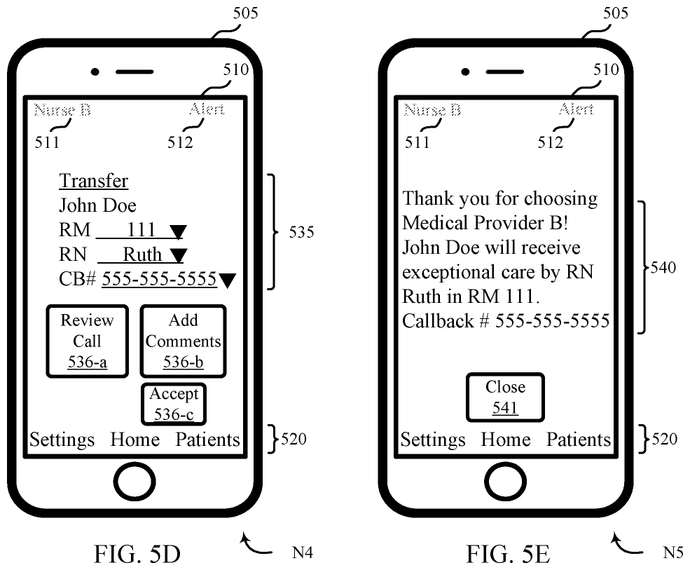

In an example aspect, the device 305 may display an alert (e.g., a notification) on the user interface 310 associated with a response to the transfer request. For example, the device 305 may display an alert via the alert indicator 312, the dialogue window 315, or both. In some aspects, the device 305 may output an audible alert in combination with the displayed alert. In an example, the device 305 may output the audible alert continuously or periodically until receiving a user input associated with the displayed alert (e.g., a user input selecting the displayed alert). In some aspects, the device 305 may receive, from the remote device 305 associated with the remote medical facility, patient transfer information including physician information, room information, additional medical personnel information, or a combination thereof. Examples of aspects of the patient transfer information are described herein with respect to examples N4 and N5 illustrated in FIGS. 5D and 5E.

FIGS. 3S and 3T illustrate examples S16 and S17 where the device 305 has received a user input selecting a displayed alert (e.g., an alert indicating an accepted transfer) with respect to the example S15-b of FIG. 3R. With reference to S16 in FIG. 3S, the device 305 may display a dialogue window 397, a “Now” button 398-a, and a “Time” button 398-b. The dialogue window 397 may include, for example, a confirmation message (e.g., an acknowledgment) including details regarding an accepted patient transfer. For example, the device 305 may receive the confirmation based on a user input from personnel (e.g., Dr. B, a nursing supervisor) at the selected medical facility (e.g., via a remote device 305 associated with the selected medical facility). In an example, the dialogue window 397 may include a message “Transfer. John Doe has been transferred via Air.”

The “Now” button 398-a may provide functionality for adding a timestamp indicating a temporal instance when the patient left the medical facility (e.g., the “Medical Provider A”). In an example, based on a user input selecting the “Now” button 398-a, the device 305 (or the server) may add a timestamp indicating the temporal instance to the patient profile. In some aspects, the device 305 or the cloud server may add a timestamp based on geospatial information associated with the medical facility (e.g., the Medical Provider A) and geospatial information associated with the vehicle transporting the patient. The device 305 or the cloud server may determine the geospatial information, for example, based on geofencing techniques.

The “Time” button 398-b may provide functionality for inputting an estimated time of arrival (ETA) associated with transporting the patient from the medical facility to the selected medical facility. In an example, based on a user input selecting the “Time” button 398-b, the device 305 may display a user interface (e.g., a keyboard) for entering an ETA. In another example, based on the user input selecting the “Time” button 398-b, the device 305 may display a scrollable interface (e.g., “Time Scroll” 398-d) for inputting the ETA. In another example, based on a user input selecting the down arrow 398-c, the device 305 may display a user interface (e.g., a dropdown menu) for selecting an ETA from a list of preconfigured durations. In some aspects, the device 305 or the cloud server may transmit an indication of the ETA (e.g., message) to the remote device 305. In some aspects, the device 305 or the cloud server may set (e.g., calculate) the ETA and automatically (e.g., based on a user setting) transmit an indication of the ETA to the remote device 305. In some example aspects, the device 305 or the cloud server may set (e.g., calculate) the ETA and provide a user interface for confirming or editing the ETA.

In an example where the device 305 received a user input selecting the “Air” button 391-a, the device 305 may calculate an ETA associated with transporting the patient from the medical facility to the selected medical facility via air transport (e.g., via helicopter). In an example where the device 305 received a user input selecting the “Ground” button 391-b, the device 305 may calculate an ETA associated with transporting the patient from the medical facility to the selected medical facility via ground transport (e.g., an emergency vehicle, for example, an ambulance). In an example where the device 305 received a user input selecting the “POV” button 391-a, the device 305 may calculate an ETA associated with transporting the patient from the medical facility to the selected medical facility via a private vehicle (e.g., via the patient's vehicle, for example, an automobile).

In some aspects, the device 305 may calculate the ETA based on geospatial information associated with the device 305, geospatial information associated with the medical facility associated with the device 305 (e.g., Medical Provider A), geospatial information associated with the remote device 305, geospatial information associated with the selected medical facility (e.g., “Medical Provider B”), geospatial information associated with the vehicle transporting the patient, or any combination thereof. In an example aspect, the device 305 may calculate the ETA utilizing mapping and navigation technologies or software (e.g., Here technologies). For example, the ETA may include an estimated travel time based on locations of the medical facilities, the locations of the devices 305, the location of the vehicle transporting the patient, a route between the locations, or any combination thereof.

In some example aspects, the device 305 may calculate the ETA based on a duration associated with preparing the patient for transfer. In some examples, the device 305 may determine the duration based on a user setting (e.g., a default duration associated with preparing a patient for transfer) or the manual user input via the “Time” button 398-b. In some aspects, the device 305 may calculate (e.g., estimate) the duration based on historical data associated with preparing patients for transfer by the medical facility. In some example aspects, the device 305 may determine the duration according to preparing patients based on different modalities (e.g., neurology, cardiology, trauma, triage, psychology, social work, ICU) or diagnoses. In an example, the ETA may include the duration for preparing the patient and the estimated travel time described herein.

In some example aspects, based on the user input indicating the ETA (e.g., via the “Now” button 398-a, the “Time” button 398-b, or the arrow 398-c), the device 305 may return the user to the main dashboard, as shown in example S17 illustrated in FIG. 3T. In some examples, the device 305 may return the user to the main dashboard, with or without displaying the “new patient” button 313 or the “existing patient” button 314. In some aspects, the device 305 may return the user to the main dashboard after automatically setting (e.g., calculating) and transmitting the ETA as described herein. In an example, the device 305 may update the dialogue window 315 with information (e.g., “John Doe, Dr. B, Medical Provider 2, Transfer ETA 45”) associated with the patient transfer.

FIGS. 4A through 4H illustrate examples R1 through R8 of a device 405 that supports medical provider to medical provider communication techniques in accordance with aspects of the present disclosure. In some examples, the device 405 may implement aspects of wireless communication system 100 or wireless communication system 200. The device 405 may be an example of aspects of the devices 115 and device 115-b as described with respect to FIGS. 1 and 2 and the device 305 (e.g., the remote device 305) as described with respect to FIGS. 3A through 3T. The device 405 may include a user interface 410. The user interface 410 and functionalities associated therewith may include examples of aspects of the user interface 310 described herein.

The examples R1 through R8 illustrated in FIGS. 4A through 4H show an example device flow associated with personnel (e.g., a hospitalist, a physician, a doctor) at a medical facility (e.g., a medical care facility, for example, a hospital, a clinic). In an example aspect, the user may be “Dr. B,” and the medical facility may be a “Medical Provider 2.” In the examples described herein, the user may access patient information associated with a patient via an application on the device 405. The device 405 may receive, via the application, a medical diagnosis for a patient from a remote device 405 associated with a medical facility (e.g., hospital, a clinic). In some aspects, the device 405 may establish, via communication protocol, a multimedia session between the device 405 and the remote device 405. Based on the multimedia session (e.g., based on a user input associated with the multimedia session), the device 405 may determine whether to initiate a patient transfer procedure for the patient. In an example aspect, the device 405 may transmit, to the remote device 405, an indication of the determination. The application on the device 405 may be an example of aspects of the application described with respect to FIGS. 1 through 3. According to examples of aspects described herein, the device 405 may execute an application supportive of medical provider to medical provider communication. The device 405 (e.g., the application) may initiate a login procedure which may prompt a user for login credentials. The login procedure may include examples of aspects of the login procedure described with respect to FIG. 3.

Referring to the example R1 illustrated in FIG. 4A, the device 405 may display a main dashboard (e.g., home dashboard) on the user interface 410. For example, the device 405 may display the main dashboard based on a successful user login (e.g., user entry of valid login credentials, user verification based on valid login credentials). The main dashboard may include, for example, a profile indicator 411, an alert indicator 412, a menu 420, a dialogue window 415, and any various additional information associated with features of the application on the device 405. The profile indicator 411, the alert indicator 412, the menu 420, the dialogue window 415, and functionalities associated therewith may include examples of aspects of the profile indicator 311, the alert indicator 312, the menu 320, and the dialogue window 315 described herein with respect to FIG. 3, respectively.

In the example illustrated in FIG. 4A, personnel (e.g., Dr. B) associated with a Medical Provider 2 (e.g., a medical care facility) is logged into the device 405, as shown at the profile indicator 411. In the example, the user has 1 unread alert, and the device 405 may indicate the unread alert using text (e.g., the term “alert”, a text format, for example, bold text, a counter (e.g., “1”), a symbol, for example, a set of asterisks, or any combination thereof at the alert indicator 412. In some aspects, the alert indicator 412 may indicate any deferred calls (e.g., deferred requests for a multimedia session). Examples of deferring requests for a multimedia session which are described herein with respect to FIG. 4B.

The dialogue window 415 may include a list of activity associated with the personnel (e.g., Dr. B) and a set of patients seen by the personnel (e.g., patients transferred to the personnel from another medical facility and examined or treated by the personnel, patients examined or treated by the personnel and transferred to another medical facility). In an example, the dialogue window 415 may include a list of incoming patients (e.g., patients transferred to the personnel from another medical facility and to be examined or treated by the personnel). The dialogue window 415 may include, for example, a message indicating the list of activity and the list of incoming patients. For example, the dialogue window 415 may include a message “Incoming Patients. John Doe. Dr. A. Med. Provider A. Via Air (ETA 45 min). John Doe. Dr. C, Med. Provider C . . . ”

In some aspects, the device 405 may provide access to patient information associated with patients (e.g., incoming patients) indicated in the dialogue window 415. In an example, based on a user input selecting a patient indicated in the dialogue window 415 (e.g., a user input selecting “John Doe” from the dialogue window 415), the device 405 may retrieve images or patient information associated with John Doe. For example, based on the user input, the device 405 may provide the user access to patient information associated with John Doe. In some aspects, the device 405 may provide the user access to information associated with a previous multimedia session associated with John Doe. The information may include, for example, images or documents (e.g., PACS images, CPACS images, test results) associated with John Doe, consultation information associated with John Doe, patient transfer information associated with John Doe, or any combination thereof, examples aspects of which are described herein with respect to FIGS. 3A through 3T.

In some aspects, the device 405 may be configured based on credentials associated with the user logged into the device 405. For example, where the user is a hospitalist (e.g., a physician) at a medical facility that only receives patients (e.g., a medical facility that does not transfer patients), the device 405 may disable features for adding new patients. In some example aspects, the device 405 may provide the list of incoming patient transfers based on modality. For example, where the user is a cardiologist, the device 405 may provide the list of incoming patient transfers based on patient transfers related to cardiology (e.g., the dialogue window 415 may include patient transfers related to cardiology and exclude patient transfers related to other modalities). In some example aspects, the device 405 may prioritize the display of patient transfers involving the personnel (e.g., patient transfers involving the user may be displayed by the device 405 at or towards the top of the dialogue window 415).

With reference to R2 in FIG. 4B, the device 405 may display a dialogue window 425, a “Defer 15 min” button 426-a, a “Defer 30 min” button 426-b, a “Decline” button 426-c, and an “Accept” button 426-d on the user interface 410. In an example, the device 405 may display the dialogue window 425, the “Defer 15 min” button 426-a, the “Defer 30 min” button 426-b, the “Decline” button 426-c, the “Accept” button 426-d, or any combination thereof, in response to receiving a call from a remote device 405 (e.g., in response to receiving a request for a multimedia session from Dr. A). The dialogue window 425 may include, for example, a message “—Incoming Call—Dr. A. Medical Provider A. Pt: John Doe.” In an example aspect where the device 405 is in a standby mode (e.g., a locked mode) or the application is closed, the device 405 may display the “Decline” button 426-c and the “Accept” button 426-d, without displaying the dialogue window 425, the “Defer 15 min” button 426-a, or the “Defer 30 min” button 426-b. In some aspects, the device 405 may provide (e.g., enable or disable, display or hide) any combination of the “Defer 15 min” button 426-a, the “Defer 30 min” button 426-b, the “Decline” button 426-c, and the “Accept” button 426-d based on user credentials, user authority, user category or classification, etc. For example, the device 405 may enable (or display) the “Defer 15 min” button 426-a and the “Defer 30 min” button 426-b and disable (or hide) the “Decline” button 426-c and the “Accept” button 426-d when a user logged into the application on the device 305 is a hospitalist (e.g., a physician) at a medical facility that receives patients. In some examples, the device 405 may enable (or display) the “Decline” button 426-c and the “Accept” button 426-d and disable (or hide)) the “Defer 15 min” button 426-a and the “Defer 30 min” button 426-b when a user logged into the application on the device 305 is nursing supervisor at the medical facility that receives patients.

The “Defer 15 min” button 426-a may provide functionality for deferring (e.g., delaying) the multimedia session for 15 minutes. The “Defer 30 min” button 426-b may provide functionality for deferring (e.g., delaying) the multimedia session request for 30 minutes. The “Decline” button 426-c may provide functionality for declining the multimedia session request. In some aspects, based on a user input selecting the “Decline” button 426-c, the device 405 may decline the multimedia session. In some aspects, based on the user input selecting the “Decline” button 426-c, the device 405 may return the user to the main dashboard, as shown in example R1 illustrated in FIG. 4A. In some aspects, based on the user input selecting the “Decline” button 426-c, the device 405 (e.g., the application) may set the Medical Provider 1 to a “divert” status, for example for diverting additional requests for multimedia sessions or for diverting transfer requests, aspects of which are described herein. In some example aspects, based on the user input selecting the “Defer 15 min” button 426-a or the “Defer 30 min” button 426-b, the device 405 may update the alert indicator 412 (e.g., “Alert (1)”) to indicate the deferred multimedia session.

In an example where the device 405 receives a subsequent call from the remote device 405 (e.g., a repeated request for a multimedia session from Dr. A) and the user again selects the “Defer 15 min” button 426-a or the “Defer 30 min” button 426-b, the device 405 may defer (e.g., delay) the multimedia session by the respective duration (e.g., 15 minutes) or a different duration (e.g., 30 minutes). In an example aspect where a multimedia session has been deferred for a duration (e.g., 15 minutes, 30 minutes), the device 405 may automatically attempt to initiate the multimedia session between medical facilities (e.g., initiate a callback from Dr. B of Medical Provider 2 to Dr. A of Medical Provider A) at or following the expiration of the duration. In some example aspects, based on a user input associated with the alert indicator 412 (e.g., a user input selecting the alert indicator 412), the device 305 may review any deferred multimedia sessions. For example, the device 305 may provide a user interface (e.g., a user prompt) for initiating the multimedia session (e.g., initiating a callback from Dr. B of Medical Provider 2 to Dr. A of Medical Provider A). In some aspects, when a multimedia session has been deferred for more than a total duration of 30 minutes (e.g., via consecutive selections of the “Defer 15 min” button 426-a, the “Defer 30 min” button 426-b, or both), the device 405 (e.g., the application) may set the Medical Provider 1 to the “divert” status.

FIGS. 4C and 4D illustrate examples R3 and R4 of a multimedia session established between the device 405 and a remote device 405 (e.g., based on a user input selecting the “Accept” button 426-d, based on an initiation of the multimedia session by the device 405). Referring to FIG. 4C, the device 405 may display multiple visual elements during a multimedia session. In the examples R3 and R4 illustrated in FIGS. 4C and 4D, the visual elements may include, for example, a dialogue window 430, an “Images” button 431, a “Record” button 432, a “Mute” button 433-a, a “Keypad” button 433-b, a “+ Call” button 433-c, a “Speaker” button 433-d, a “Video” button 433-e, an “Audio” button 433-f, a “Consult” button 433-g, a “Transfer” button 433-h, a “Flip” button 433-I, a window 435-a, and a preview window 435-b.

The dialogue window 430, the “Images” button 431, the “Record” button 432, the “Mute” button 433-a, the “Keypad” button 433-b, the “+ Call” button 433-c, the “Speaker” button 433-d, the “Video” button 433-e, the “Audio” button 433-f, the “Consult” button 433-g, the “Transfer” button 433-h, the “Flip” button 433-I, the window 435-a, the preview window 435-b, and functionalities associated therewith may include examples of aspects of the dialogue window 370, the “Images” button 371, the “Record” button 372, the “Mute” button 373-a, the “Keypad” button 373-b, the “+ Call” button 373-c, the “Speaker” button 373-d, the “Video” button 373-e, the “Audio” button 373-f, the “Consult” button 373-g, the “Transfer” button 373-h, the “Flip” button 373-I, the window 375-a, and the preview window 375-b described with respect to examples S9 and S10 illustrated in FIGS. 3K and 3L, respectively. In some aspects, the device 405 may hide (e.g., refrain from displaying) the “+ Call” button 433-c.

In an example aspect, the “Images” button 431 may provide functionality for displaying images received by the device 405 from the remote device 405. The device 405 may display the images based on a user input selecting the “Images” button 431. In an example, the device 405 may receive the images retrieved, selected, and shared by a remote device 405 (e.g., the device 305 in the examples S6-a and S7-a described with respect to FIGS. 3F and 3G, e.g., “Neurology” modality). In another example, the device 405 may display the images captured (e.g., scanned), uploaded, and shared by the remote device 405 (e.g., the remote device 305 in the examples S6-b and S7-b described with respect to FIGS. 3H and 3I, e.g., “Cardiology” modality).

With reference to R5 in FIG. 4E, the device 405 may display the retrieved images. In some aspects, the device 405 may provide a user interface via which a user may scroll (e.g., via a swipe gesture) and select between the images shared by the remote device 405. In the example R5 illustrated in FIG. 4E, the images may include images 440-b through 440-e. In some aspects, the device may display an enlarged view or preview image (e.g., image 440-a) of a selected image. For example, based on a user input scrolling to or selecting the image 440-b, the device 405 may display the image 440-a, where the image 440-a is an enlarged view of the image 440-b.

With reference to R6 in FIG. 4F, the device 405 may display a dialogue window 445, a “Review Call” button 446-a, an “Add Comments” button 446-b, and a “Close” button 446-c based on a user input, for example, based on a user input (e.g., by “Dr. B”) selecting the “Consult” button 433-g with respect to any of FIG. 4C or 4D, a user input (e.g., by “Dr. A”) selecting the “Consult” button 373-g with respect to any of FIGS. 3K through 3L, or both. The dialogue window 445, the “Review Call” button 446-a, the “Add Comments” button 446-b, and the “Close” button 446-c and functionalities associated therewith may include examples of aspects of the dialogue window 380, the “Review Call” button 381-a, the “Add Comments” button 381-b, and the “Close” button 381-c described with respect to example S11 illustrated in FIG. 3M, respectively. In an example, the dialogue window 445 may include a message “Consult. John Doe . . . Dr. B. Medical Provider 2” associated with the multimedia session between the device 405 and the remote device 405. In an example aspect, based on a user input selecting the “Close” button 446-c with respect to FIG. 4F, the device 405 may return the user to the main dashboard described with respect to FIG. 4A. In an example, the device 405 may update the dialogue window 415 with information (e.g., “John Doe . . . Dr. A. Medical Provider A. Consult”) associated with the multimedia session (e.g., the consult). The updates of the dialogue window 415 may include examples of aspects of the updating of the dialogue window 315 with respect to FIG. 3O.

With reference to R7 in FIG. 4G, the device 405 may display a dialogue window 450, a “Record Audio” button 451-a, an “Accept” button 451-b, a text window 451-c, and a keyboard 451-d based on a user input (e.g., by “Dr. B”) selecting the “Add Comments” button 381-b. The dialogue window 450, the “Record Audio” button 451-a, the “Accept” button 451-b, the text window 451-c, the keyboard 451-d, and functionalities associated therewith may include examples of aspects of the dialogue window 385, the “Record Audio” button 386-a, the “Accept” button 386-b, the text window 386-c, and the keyboard 386-d described with respect to example S12 illustrated in FIG. 3N, respectively.

With reference to R8 in FIG. 4H, the device 405 may output (e.g., submit, transmit) a response to a transfer request received by the device 405 from a remote device 405. In an example, the device 405 may be associated with a medical facility (e.g., the Medical Provider 2). The device 405 may receive a transfer request output (e.g., submitted, transmitted) by the remote device 405 associated with another medical facility (e.g., the “Medical Provider A”). In some aspects, the device 405 may receive the transfer request from the remote device 405, a cloud server, or both. Based on a user input from personnel (e.g., Dr. B, a nursing supervisor) at the medical facility (e.g., via the device 405), the device 405 may output (e.g., submit, transmit) a response associated with the transfer request (e.g., a response accepting, denying, or delaying the transfer).

With respect to R8 in FIG. 4H, the device 405 (e.g., based on a user input by Dr. B or a nursing supervisor via the device 405) has accepted and output a response to the transfer request. The device 405 may display a dialogue window 455 and a “Close” button 456. The dialogue window 455 may include, for example, a confirmation message (e.g., an acknowledgment) including details regarding the accepted patient transfer. The confirmation message may include, for example, a transportation type associated with the patient transfer, an ETA associated with the patient transfer, patient information (e.g., patient name, date of birth, diagnosis), hospitalist associated with requesting the transfer (e.g., Dr. A), medical facility associated with requesting the transfer (e.g., Medical Provider A), or a combination thereof. In an example, the dialogue window 455 may include a message “Your transfer is enroute via air (ETA 45 min). Pt: John Doe . . . Dr. A. Medical Provider A.”

In an example aspect, the device 405 may display an alert (e.g., a notification) associated with the accepted transfer on the user interface 410. For example, the device 405 may display an alert via the alert indicator 412, the dialogue window 415, or both. In some aspects, the device 405 may output an audible alert in combination with the displayed alert. In an example, the device 405 may output the audible alert continuously or periodically until receiving a user input associated with the displayed alert (e.g., a user input selecting the displayed alert).

According to examples of the aspects described herein, multiple devices 405 involved in the accepted transfer (e.g., multiple devices 405 associated with medical facilities involved in the transfer) may receive and display the alert associated with the accepted transfer. For example, a device 405 logged into by a nursing supervisor associated with a medical facility accepting the patient transfer (e.g., the Medical Provider 2), a device 405 logged into by a hospitalist (e.g., a cardiologist, a neurologist) associated with medical facility accepting the patient transfer (e.g., the Medical Provider 2), and a remote device 405 logged into by a hospitalist (e.g., a cardiologist, a neurologist) associated with a medical facility requesting the patient transfer (e.g., the Medical Provider A) may each display the alert (e.g., the notification) associated with the accepted transfer. In an example, the devices 405 may receive the alert from a cloud server.

The remote device 405 may display information associated with the nursing supervisor (e.g., an accepting nurse) associated with a medical facility accepting the patient transfer (e.g., the Medical Provider 2). In some examples, the remote device 405 may display a selectable option that enables the remote device 405 to contact (e.g., via an audio call, a video call, or a message) the nursing supervisor associated with a medical facility accepting the patient transfer. For example, the remote device 405 may establish a connection (e.g., a multimedia session) with the device 405 logged into by the nursing supervisor associated with the medical facility accepting the patient transfer (e.g., the Medical Provider 2) or a personal device of the nursing supervisor. As such, the remote device 405 may provide a mechanism to contact (e.g., call-back) the accepting nurse directly.

In some aspects, devices of users indirectly involved in the accepted transfer may also receive the alert associated with the accepted transfer. For example, an admissions clerk at a medical facility receiving the transferred patient may receive an email, a desktop push notification, or both based on the accepted transfer. In some aspects, the admissions clerk may receive status changes associated with the transfer (e.g., travel updates associated with the patient, for example, when the patient is on route, when the transferred patient arrives). In some aspects, the email, desktop push notification, or both may include a link (e.g., a link to a web portal) for accessing details of the transfer (e.g., the transfer status).

In an alternative example, the device 405 (e.g., based on a user input by Dr. B or a nursing supervisor via the device 405) may select an input denying the transfer request. Based on the user input denying the transfer, the remote device 405 requesting the transfer may display, in the dialogue window 455, a denial message including details regarding the denied patient transfer. In some aspects, the denial message may include a prompt for diverting the patient to a different medical facility. For example, the denial message may include a medical facility recommended by Dr. B or the nursing supervisor at the Medical Provider 2.

In an example aspect, the device 405 may set the Medical Provider 1 to a “divert” status. For example, based on the user input denying the transfer request, the device 405 may automatically set the Medical Provider 1 to a “divert” status for a duration (e.g., 2 hours). In some examples, based on the user input denying the transfer request, the device 405 may display a virtual button for enabling the “divert” status. In an example, the device 405 may display a user interface for selecting the duration for the “divert” status. In some aspects, the device 405 may display a user interface for inputting a reason or comments associated with the “divert” status. In some example aspects, the device 405 may communicate the “divert” status to a third party (e.g., a state medical board) which may notify other medical providers of the “divert” status. Based on a user input selecting the “Close” button 456, the device 405 may return the user to the main dashboard described with respect to FIG. 4A. In an example, the device 405 may update the dialogue window 415 with information associated with the accepted patient transfer or the denied patient transfer. The updates of the dialogue window 415 may include examples of aspects of the updating of the dialogue window 315 with respect to FIG. 3O.

FIGS. 5A through 5E illustrate examples N1 through N5 of a device 505 that supports medical provider to medical provider communication techniques in accordance with aspects of the present disclosure. In some examples, the device 505 may implement aspects of wireless communication system 100. The device 505 may be an example of aspects of the devices 115 as described with respect to FIGS. 1 and 2, the device 305 (e.g., the remote device 305) as described with respect to FIGS. 3A through 3T, and the device 405 (e.g., the remote device 405) as described with respect to FIGS. 4A through 4H. The device 505 may include a user interface 510. The user interface 510 and functionalities associated therewith may include examples of aspects of the user interface 310 and the user interface 410 described herein.

The examples N1 through N5 illustrated in FIGS. 5A through 5E show an example device flow associated with personnel (e.g., a nursing supervisor) at a medical facility. In an example aspect, the user may be “Nurse B” and the medical facility may be “Medical Provider 2.” In the examples described herein, the user may access patient information associated with a patient via an application on the device 505. The device 505 may receive and display, via the application, information indicating incoming patient transfers from other medical facilities (e.g., Medical Provider A). In some aspects, the device 505 may receive and display recent activity for modalities (e.g., trauma, triage, neurology, cardiology) provided by the medical facility (e.g., Medical Provider 2). In some aspects, the device 505 may receive a medical diagnosis for a patient from a remote device 505. In some aspects, the device 505 may determine whether to initiate a patient transfer procedure for the patient. In an example aspect, the device 505 may transmit, to the remote device 505, an indication of the determination. The application may be an example of aspects of the application on the devices 115, 305, and 405 described with respect to FIGS. 1 through 4.

According to examples of aspects described herein, the device 505 may execute an application supportive of medical provider to medical provider communication. The device 505 (e.g., the application) may initiate a login procedure which may prompt a user for login credentials. The login procedure may include examples of aspects of the login procedure described with respect to FIGS. 3 and 4. In some aspects, the device 505 may provide functions for accepting, denying, or deferring a request for a multimedia session. For example, based on a user input, the device 505 may accept, deny, or defer a request for a multimedia session. In some aspects, the device 405 may provide all or a combination of the functions for accepting, denying, or deferring the request for the multimedia session based on user credentials, user authority, user category or classification, etc. Aspects of the device 505 accepting, denying, or deferring a request for a multimedia session may include examples of aspects described with respect to the device 405 described with respect to FIG. 4B. In some aspects, the device 505 may be configured based on credentials associated with the user logged into the device 505. In an example, where the user is a nurse supervisor, and the user selects a user input accepting a request for a multimedia session (e.g., answers a call), the device 505 may enable audio streaming (but not video streaming) for the multimedia session.

With reference to N1 in FIG. 5A, the device 505 may display a main dashboard (e.g., home dashboard) on the user interface 510. For example, the device 505 may display the main dashboard based on a successful user login (e.g., user entry of valid login credentials, user verification based on valid login credentials). The main dashboard may include, for example, a profile indicator 511, an alert indicator 512, a menu 520, a dialogue window 515, and any various additional information associated with features of the application on the device 505. The profile indicator 511, the alert indicator 512, the menu 520, the dialogue window 515, and functionalities associated therewith may include examples of aspects of the profile indicator 311, the alert indicator 312, the menu 320, and the dialogue window 315 described herein with respect to FIG. 3, respectively. In some examples, the profile indicator 511, the alert indicator 512, the menu 520, the dialogue window 515, and functionalities associated therewith may include examples of aspects of the profile indicator 411, the alert indicator 412, the menu 420, and the dialogue window 415 described herein with respect to FIG. 4, respectively.

With reference to FIG. 5A, a user “Nurse B” associated with a Medical Provider 2 (e.g., a medical care facility) is logged into the device 505, as shown at the profile indicator 511. In the example, the user has no unread alerts. The dialogue window 515 may include a list of activity associated with recent activity for modalities (e.g., trauma, triage, neurology, cardiology) provided by the medical facility (e.g., Medical Provider 2). In an example, the dialogue window 515 may include a list of incoming patients (e.g., patients transferred to the medical facility for examination or treatment). The dialogue window 515 may include, for example, a message “Incoming Patients. John Doe. Dr. A. Med. Provider A. Dr. B. Rm 111. Enroute via Air (ETA 45 min).”

With reference to N2 in FIG. 5B, the device 505 indicates the user has an unread alert. The device 505 may indicate the unread alert using text (e.g., the term “alert”, a text format, for example, bold text, a counter (e.g., “1”), a symbol, for example, a set of asterisks, or any combination thereof at the alert indicator 512. In the example N2 illustrated in FIG. 3B, the alert may be associated with an incoming transfer request. In some aspects, properties associated with the alerts (e.g., volume associated with an audio notification) may be configured based on urgency level associated with the alert.

The device 505 may display an “Alert” button 525 providing functionality for reviewing the incoming patient transfer request. In some aspects, based on a user input selecting the “Alert” button 525, the device 505 may display information associated with the incoming patient transfer request. The information may include details regarding the patient transfer request. In some aspects, the information may include a patient profile and relevant contact information associated with the patient (e.g., phone number, email address). In some aspects, in an example where the device 505 has not received a response from the user, the device 505 may display a notification inquiring whether to maintain an “On Divert” status (or a “Full Divert” status). In an example aspect where the device 505 does not receive a user input responding to the notification, for example, within a preconfigured duration (e.g., 15 minutes), the device 505 may disable the “On Divert” status (or the “Full Divert” status). Examples of aspects of the “On Divert” status and the “Full Divert” status are described herein with respect to the “Reject” button 532.

In some aspects, the device 505 may output an audible alert in combination with the displayed alert. In an example, the device 505 may output the displayed alert or the audible alert continuously or periodically until receiving a user input associated with an unread alert (e.g., a user input selecting the unread alert). In some examples, the device 505 may output the displayed alert or the audible alert every 5 minutes until receiving a user input associated with the unread alert. In some aspects, if outputting an alert two times without receiving a user response, the device 505 may output the alert a third time in combination with transmitting an email communication to the user, the medical facility, or administration staff associated with the application on the device 505. The email communication may include a notification of the missed alerts.

With reference to N3 in FIG. 5C, the device 505 has received a user input selecting the “Alert” button 525. The device 505 may display a dialogue window 530, an “Accept” button 531, and a “Reject” button 532. The dialogue window 530 may include, for example, a message including details regarding the patient transfer request. For example, the message may be a “Transfer” message including the name of the patient, a diagnosis associated with the patient, users associated with transfer (e.g., Dr. A, Dr. B) or the transfer, medical facilities associated with the users (e.g., Medical Provider A, Medical Provider 2), a diagnosis associated with the patient, or any combination thereof. In an example, the dialogue window 530 may include a message “Transfer. John Doe . . . Dr. A, Med. Provider A. Dr. B. Stroke.”

The “Accept” button 531 may provide functionality for accepting the patient transfer request. The “Reject” button 532 may provide functionality for rejecting the patient transfer request. In some aspects, based on a user input selecting the “Reject” button 532, the device 505 may display a prompt for adding comments associated with rejecting the patient transfer request (e.g., a reason the user rejected the patient transfer request). In an example aspect, the device 505 may provide an option for the user to indicate whether the medical facility (e.g., Medical Provider 2) is not accepting patient transfer requests for the modality associated with the rejected patient transfer request (e.g., an “On Divert” status). In some example aspects, the device 505 may provide an option for the user to indicate whether the medical facility is not accepting patient transfer requests for all modalities (e.g., a “Full Divert” status).

In an example, based on a user input indicating an “On Divert” status for a modality, the device 505 may automatically divert patient transfer requests associated with the modality. In some aspects, after a preconfigured duration (e.g., 1 hour and 45 minutes) has elapsed, the device 505 may display a notification inquiring whether to maintain the “On Divert” status (or the “Full Divert” status). In an example aspect where the device 505 does not receive a user input responding to the notification, for example, within a preconfigured duration (e.g., 15 minutes), the device 505 may disable the “On Divert” status (or the “Full Divert” status).

With reference to N4 in FIG. 5D, the device 505 has received a user input selecting the “Accept” button 531. The device 505 may display a dialogue window 535, a “Review Call” button 536-a, an “Add Comments” button 536-b, and an “Accept” button 536-c. The dialogue window 535 may include a user interface for assigning a room (e.g., RM 111), a nurse (e.g., Ruth), and a contact information (e.g., a callback telephone number 555-555-5555) to the patient profile associated with the patient transfer request. The “Review Call” button 536-a may include examples of aspects of the “Review Call” button 381-a described with respect to FIG. 3M and the “Review Call” button 446-a described with respect to FIG. 4F. The “Add Comments” button 536-b may include examples of aspects of the “Add Comments” button 381-b described with respect to FIG. 3M and the “Add Comments” button 446-b described with respect to FIG. 4F.

With reference to N5 in FIG. 5E, the device 505 has received a user input selecting the “Accept” button 536-c. The device 505 may display a dialogue window 540 and a “Close” button 541. In an example, the dialogue window 540 may include a confirmation message including the assigned room, nurse, and contact number described with respect to FIG. 5D. For example, the dialogue window 540 may include a confirmation message “Thank you for choosing Medical Provider B! John Doe will receive exceptional care by RN Ruth in RM 111. Callback #555-555-5555.” In some aspects, all or some of the devices 305, 405, or 505 associated with users (e.g., Dr. A, Dr. B) involved in the patient transfer may receive and display the confirmation message. In some example aspects, based on a user input selecting the “Close” button 541, the device 505 may return the user to the main dashboard (e.g., FIG. 5A).

Examples of aspects are described herein with respect to personnel (e.g., a physician, a hospitalist) at a medical facility, personnel (e.g., a physician, a hospitalist) at a different medical facility, and other personnel (e.g., a nursing supervisor, medical staff) at the different medical facility. The examples of aspects described herein may be applied to any personnel. For example, the devices 115, 305, 405, and 505 may be accessed by personnel such as hospitalists, physicians, nurses, nursing supervisors, support staff (e.g., admissions clerks), etc. The devices 115, 305, 405, and 505 may be configured to provide any combination of features described herein, for example, based on user credentials, user authority, user category or classification, etc. The communications described herein may include, for example, short message service (SMS) communications, in-application messaging, email messaging, and the like.

In some aspects, the devices 115, 305, 405, and 505 may be configured to provide features for adding, updating, or modifying contact information associated with the medical facility described herein. For example, the devices 115, 305, 405, and 505 may include features for adding, updating, or modifying the phone number or email address used for an admissions desk at a receiving medical facility. In some aspects, the buttons illustrated herein as displayed on the user interfaces 310, 410, and 510 may include any combination of text, graphics, or icons.

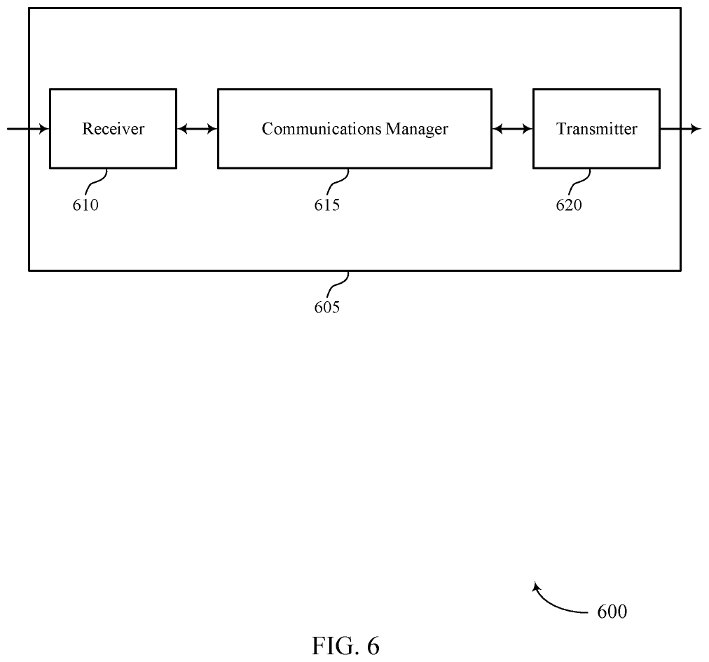

FIG. 6 shows a block diagram 600 of a device 605 that supports medical provider to medical provider communications in accordance with aspects of the present disclosure. The device 605 may be an example of aspects of a device as described herein. The device 605 may include a receiver 610, a communications manager 615, and a transmitter 620. The device 605 may also include a processor. Each of these components may be in communication with one another (e.g., via one or more buses).

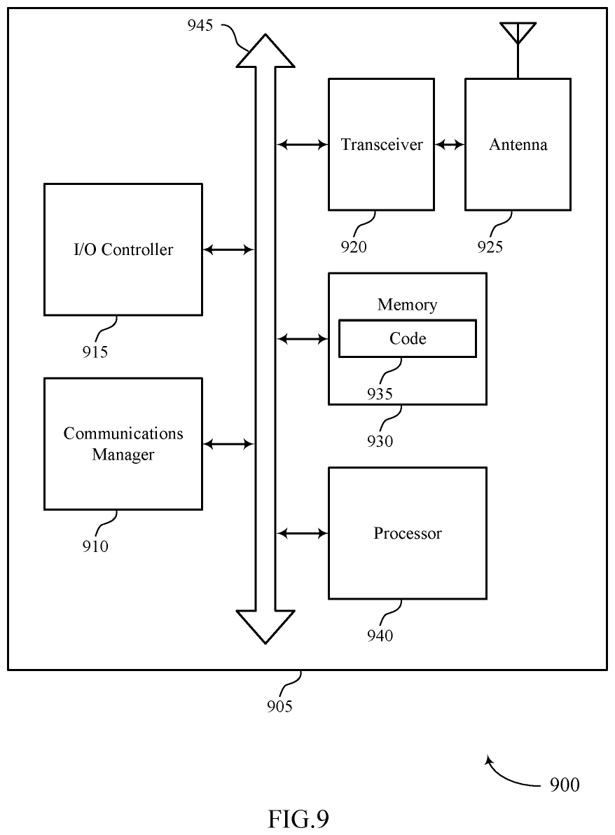

The receiver 610 may receive information such as packets, user data, or control information associated with various information channels (e.g., control channels, data channels, and information related to medical provider to medical provider communications, etc.). Information may be passed on to other components of the device 605. The receiver 610 may be an example of aspects of the transceiver 920 described with reference to FIG. 9. The receiver 610 may utilize a single antenna or a set of antennas.

The communications manager 615 may access patient information associated with a patient via an application on the device 605, select, via the application, a remote medical provider from a list of remote medical providers or a medical diagnosis for the patient, or both, based on the patient information, establish, using a communication protocol, a multimedia session between the device 605 and a remote device associated with the remote medical provider, determine whether to initiate a patient transfer procedure for the patient based on the multimedia session, and transmit, to the remote device, an indication of determining whether to initiate the patient transfer procedure for the patient. The communications manager 615 may be an example of aspects of the communications manager 910 described herein.

The communications manager 615, or its sub-components, may be implemented in hardware, code (e.g., software or firmware) executed by a processor, or any combination thereof. If implemented in code executed by a processor, the functions of the communications manager 615, or its sub-components may be executed by a general-purpose processor, a DSP, an application-specific integrated circuit (ASIC), a FPGA or other programmable logic device, discrete gate or transistor logic, discrete hardware components, or any combination thereof designed to perform the functions described in the present disclosure.

The communications manager 615, or its sub-components, may be physically located at various positions, including being distributed such that portions of functions are implemented at different physical locations by one or more physical components. In some examples, the communications manager 615, or its sub-components, may be a separate and distinct component in accordance with various aspects of the present disclosure. In some examples, the communications manager 615, or its sub-components, may be combined with one or more other hardware components, including but not limited to an input/output (I/O) component, a transceiver, a network server, another computing device, one or more other components described in the present disclosure, or a combination thereof in accordance with various aspects of the present disclosure.

The transmitter 620 may transmit signals generated by other components of the device 605. In some examples, the transmitter 620 may be collocated with a receiver 610 in a transceiver component. For example, the transmitter 620 may be an example of aspects of the transceiver 920 described with reference to FIG. 9. The transmitter 620 may utilize a single antenna or a set of antennas.

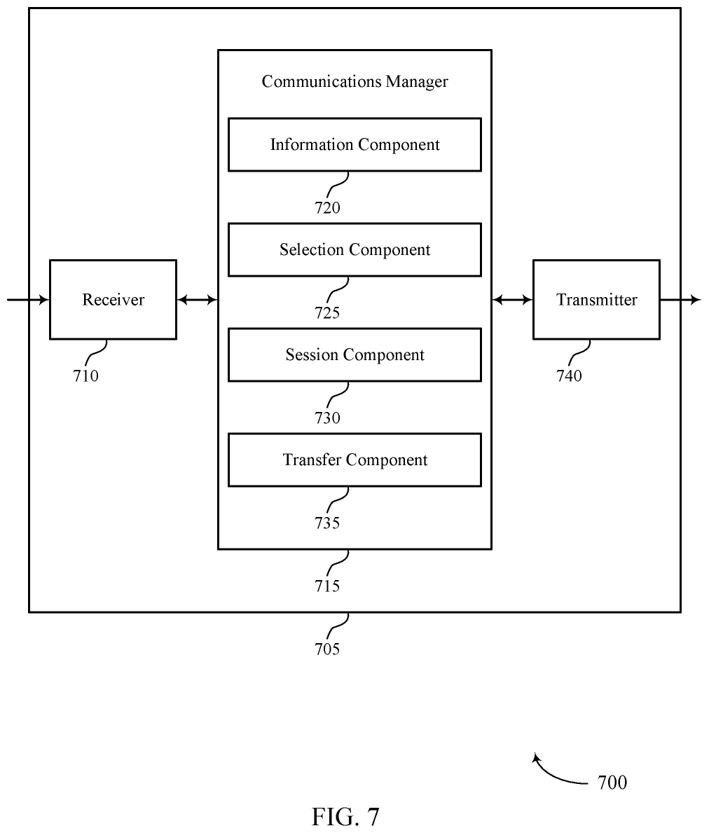

FIG. 7 shows a block diagram 700 of a device 705 that supports medical provider to medical provider communications in accordance with aspects of the present disclosure. The device 705 may be an example of aspects of a device 605 or a device 115 as described herein. The device 705 may include a receiver 710, a communications manager 715, and a transmitter 740. The device 705 may also include a processor. Each of these components may be in communication with one another (e.g., via one or more buses). One or more of these components may be means for supporting medical provider to medical provider communications in accordance with aspects of the present disclosure.

The receiver 710 may receive information such as packets, user data, or control information associated with various information channels (e.g., control channels, data channels, and information related to medical provider to medical provider communications, etc.). Information may be passed on to other components of the device 705. The receiver 710 may be an example of aspects of the transceiver 920 described with reference to FIG. 9. The receiver 710 may utilize a single antenna or a set of antennas.

The communications manager 715 may be an example of aspects of the communications manager 615 as described herein. The communications manager 715 may include an information component 720, a selection component 725, a session component 730, and a transfer component 735. The communications manager 715 may be an example of aspects of the communications manager 910 described herein.

The information component 720 may access patient information associated with a patient via an application on the device 705. The selection component 725 may select, via the application, a remote medical provider from a list of remote medical providers or a medical diagnosis for the patient, or both, based on the patient information. The session component 730 may establish, using a communication protocol, a multimedia session between the device 705 and a remote device associated with the remote medical provider. The transfer component 735 may determine whether to initiate a patient transfer procedure for the patient based on the multimedia session and transmit, to the remote device, an indication of determining whether to initiate the patient transfer procedure for the patient.

The transmitter 740 may transmit signals generated by other components of the device 705. In some examples, the transmitter 740 may be collocated with a receiver 710 in a transceiver component. For example, the transmitter 740 may be an example of aspects of the transceiver 920 described with reference to FIG. 9. The transmitter 740 may utilize a single antenna or a set of antennas.

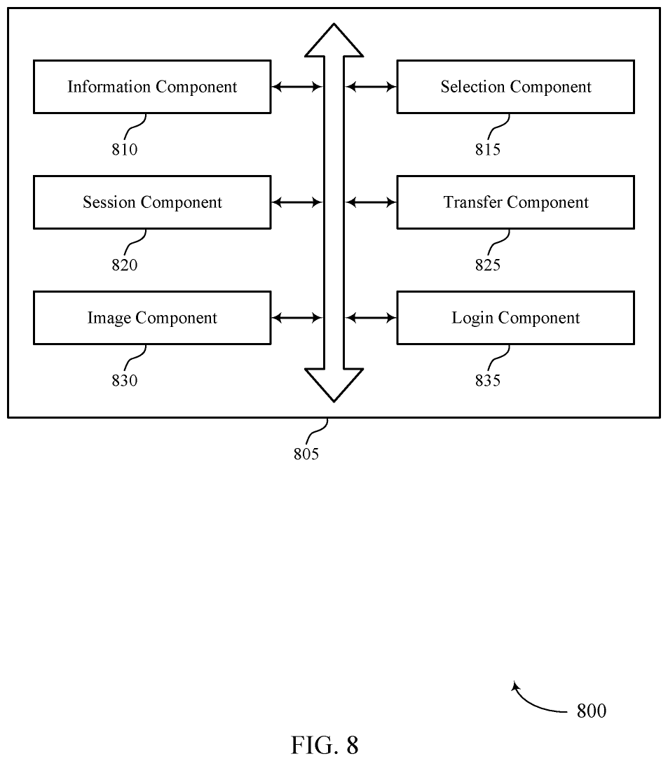

FIG. 8 shows a block diagram 800 of a communications manager 805 that supports medical provider to medical provider communications in accordance with aspects of the present disclosure. The communications manager 805 may be an example of aspects of a communications manager 615, a communications manager 715, or a communications manager 910 described herein. The communications manager 805 may include an information component 810, a selection component 815, a session component 820, a transfer component 825, an image component 830, and a login component 835. Each of these components may communicate, directly or indirectly, with one another (e.g., via one or more buses).

The information component 810 may access patient information associated with a patient via an application on a device. In some examples, the information component 810 may identify a patient account corresponding to the patient. In some examples, accessing the patient information may include retrieving the patient information from the patient account via the application running on the device or a server, or any combination thereof. In some examples, the information component 810 may generate a patient account corresponding to the patient associated with the patient information. In some examples, the information component 1910 may store the patient account in a database remote or local to the device or a server, or any combination thereof. In some examples, the information component 810 may compile the patient information based on one or more information elements.

In some examples, the information component 810 may determine a medical diagnosis for the patient based on the patient information. In some cases, the medical diagnosis for the patient includes a cardiological medical diagnosis, a neurological medical diagnosis, a trauma medical diagnosis, or a triage medical diagnosis, or any combination thereof. In some cases, a patient transfer procedure is associated with a medical diagnosis for the patient, the medical diagnosis including a cardiological medical diagnosis, a neurological medical diagnosis, a trauma medical diagnosis, or a triage medical diagnosis, or any combination thereof.

The selection component 815 may select, via the application, a remote medical provider from a list of remote medical providers or a medical diagnosis for the patient, or both, based on the patient information. In some examples, the selection component 815 may provide, via an interface of the application, a selectable option to consult the patient at a local medical provider or transfer the patient to the remote medical provider based on terminating the multimedia session. In some examples, the selection component 815 may receive an input associated with the selectable option, where the input indicates a selection to transfer the patient to the remote medical provider. In some examples, the selection component 815 may receive an input associated with the selectable option, where the input indicates a selection to consult the patient at the local medical provider.

The selection component 815 may determine the list of remote medical providers based on the patient information or the medical diagnosis for the patient, or both. In some examples, the selection component 815 may determine one or more locations associated with one or more remote medical providers, including the remote medical provider, in the list of remote medical providers that satisfy a location threshold relative to the patient. In some examples, the selection component 815 may select the remote medical provider from the list of remote medical providers based on the location threshold relative to the patient. In some examples, the selection component 815 may provide, via an interface of an application on the device, a selectable option to consult the patient at the remote medical provider or transfer the patient to a local medical provider based on terminating the multimedia session. In some examples, the selection component 815 may receive an input associated with the selectable option, where the input indicates a selection to transfer the patient to the local medical provider.

The session component 820 may establish, using a communication protocol, a multimedia session between the device and a remote device associated with the remote medical provider. In some examples, the session component 820 may establish, using a communication protocol, a multimedia session between a device and a remote device associated with a remote medical provider. In some examples, the session component 820 may terminate the multimedia session. In some examples, the session component 820 may transcribe the multimedia session based on the selection to consult the patient at the local medical provider. In some examples, the session component 820 may provide a report of the transcribed multimedia session to the local medical provider or the remote medical provider, or both.

In some examples, the session component 820 may transmit, to the remote device associated with the remote medical provider, a request message to establish the multimedia session, the request message including session information for the multimedia session. In some examples, the session component 820 may receive, from the remote device associated with the remote medical provider, a response message based on the request message, the response message including a second indication of accepting, delaying, or declining the request message to establish the multimedia session, the session information including the patient information or the medical diagnosis for the patient, or both. In some examples, the session component 820 may establish the multimedia session between the device and the remote device based on the response message.

In some examples, the session component 820 may include a second indication of the medical diagnosis in session information for the multimedia session. In some examples, the session component 820 may terminate the multimedia session. In some examples, the session component 820 may receive, from the remote device associated with the remote medical provider, a request message to establish the multimedia session between the device and the remote device, the request message including session information for the multimedia session.

In some examples, the session component 820 may transmit, to the remote device associated with the remote medical provider, a response message based on the request message, the response message including a second indication of accepting, delaying, or declining the request message to establish the multimedia session, the session information including patient information associated with the patient or a medical diagnosis for the patient, or both. In some examples, the session component 820 may establish the multimedia session between the device and the remote device based on the response message. In some cases, the multimedia session includes an audio session or a video session, or both.

The transfer component 825 may determine whether to initiate a patient transfer procedure for the patient based on the multimedia session. In some examples, the transfer component 825 may transmit, to the remote device, an indication of determining whether to initiate the patient transfer procedure for the patient. In some examples, the transfer component 825 may determine whether to initiate a patient transfer procedure for a patient based on the multimedia session. In some examples, the transfer component 825 may transmit, to the remote device, an indication of determining whether to initiate the patient transfer procedure for the patient. In some examples, the transfer component 825 may initiate the patient transfer procedure based on the selection to transfer the patient to the remote medical provider. In some examples, the transfer component 825 may transmit, to the remote device, the indication based on the initiating.

In some examples, the transfer component 825 may transmit a patient transfer notification to the remote device associated with the remote medical provider, the patient transfer notification including the patient information. In some examples, the transfer component 825 may receive, at the device, an acknowledgment to transfer the patient to the remote medical provider based on the patient transfer notification. In some examples, the transfer component 825 may receive, from the remote device associated with the remote medical provider, patient transfer information including physician information, room information, additional medical personnel information, or any combination thereof.

In some examples, the transfer component 825 may initiate the patient transfer procedure based on the selection to transfer the patient to the local medical provider. In some examples, the transfer component 825 may transmit, to the remote device, the indication based on the initiating. In some examples, the transfer component 825 may receive a patient transfer notification from the remote device associated with the remote medical provider, the patient transfer notification including patient information associated with the patient. In some examples, the transfer component 825 may transmit, at the device, an acknowledgment to transfer the patient to the local medical provider based on the patient transfer notification. In some examples, the transfer component 825 may transmit, to the remote device associated with the remote medical provider, patient transfer information including physician information, room information, additional medical personnel information, or any combination thereof.

The image component 830 may retrieve one or more images corresponding to the medical diagnosis for the patient. In some examples, the image component 830 may include, in the session information, the one or more images corresponding to the medical diagnosis for the patient. In some examples, the image component 830 may capture an image of a document including the patient information via a camera of the device. In some examples, the image component 830 may determine one or more information elements of the document using an optical character recognition operation on the document. In some examples, the image component 830 may receive, in the session information, one or more images corresponding to the medical diagnosis for the patient.

The login component 835 may determine a request to access the application running on the device. In some examples, the login component 835 may provide the access to the application running on the device based on an authentication procedure. In some examples, the login component 835 may receive credential information via the device, the credential information including a username and a password, a facial recognition, a voice recognition, a biosignature recognition, or any combination thereof. In some examples, the login component 835 may authenticate the device based on the credential information.

In some examples, the login component 835 may receive first credential information from via the device. In some examples, the login component 835 may authenticate the first credential information. In some examples, the login component 835 may receive second credential information via the device based on authenticating the first credential information. In some examples, the login component 835 may authenticate the second credential information. In some aspects, the first credential information or the second credential information, or both may include a username and a password, a facial recognition, a voice recognition, a biosignature recognition, or any combination thereof.

FIG. 9 shows a diagram of a system 900 including a device 905 that supports medical provider to medical provider communications in accordance with aspects of the present disclosure. The device 905 may be an example of or include the components of device 605, device 705, or a device as described herein. The device 905 may include components for bi-directional voice and data communications including components for transmitting and receiving communications, including a communications manager 910, an I/O controller 915, a transceiver 920, an antenna 925, memory 930, a processor 940, and a coding manager 950. These components may be in electronic communication via one or more buses (e.g., bus 945).

The communications manager 910 may access patient information associated with a patient via an application on the device 905, select, via the application, a remote medical provider from a list of remote medical providers or a medical diagnosis for the patient, or both, based on the patient information, establish, using a communication protocol, a multimedia session between the device 905 and a remote device associated with the remote medical provider, determine whether to initiate a patient transfer procedure for the patient based on the multimedia session, and transmit, to the remote device, an indication of determining whether to initiate the patient transfer procedure for the patient.

The I/O controller 915 may manage input and output signals for the device 905. The I/O controller 915 may also manage peripherals not integrated into the device 905. In some cases, the I/O controller 915 may represent a physical connection or port to an external peripheral. In some cases, the I/O controller 915 may utilize an operating system such as iOS®, ANDROID®, MS-DOS®, MS-WINDOWS®, OS/2®, UNIX®, LINUX®, or another known operating system. In other cases, the I/O controller 915 may represent or interact with a modem, a keyboard, a mouse, a touchscreen, or a similar device. In some cases, the I/O controller 915 may be implemented as part of a processor. In some cases, a user may interact with the device 905 via the I/O controller 915 or via hardware components controlled by the I/O controller 915.

The transceiver 920 may communicate bi-directionally, via one or more antennas, wired, or wireless links as described above. For example, the transceiver 920 may represent a wireless transceiver and may communicate bi-directionally with another wireless transceiver. The transceiver 920 may also include a modem to modulate the packets and provide the modulated packets to the antennas for transmission, and to demodulate packets received from the antennas. In some cases, the device 905 may include a single antenna 925. However, in some cases, the device 905 may have more than one antenna 925, which may be capable of concurrently transmitting or receiving multiple transmissions.

The memory 930 may include RAM and ROM. The memory 930 may store computer-readable, computer-executable code 935 including instructions that, when executed, cause the processor to perform various functions described herein. In some cases, the memory 930 may contain, among other things, a BIOS which may control basic hardware or software operation such as the interaction with peripheral components or devices.

The code 935 may include instructions to implement aspects of the present disclosure, including instructions to support wireless communications. The code 935 may be stored in a non-transitory computer-readable medium such as system memory or other type of memory. In some cases, the code 935 may not be directly executable by the processor 940 but may cause a computer (e.g., when compiled and executed) to perform functions described herein.

The processor 940 may include an intelligent hardware device, (e.g., a general-purpose processor, a DSP, a CPU, a microcontroller, an ASIC, an FPGA, a programmable logic device, a discrete gate or transistor logic component, a discrete hardware component, or any combination thereof). In some cases, the processor 940 may be configured to operate a memory array using a memory controller. In other cases, a memory controller may be integrated into the processor 940. The processor 940 may be configured to execute computer-readable instructions stored in a memory (e.g., the memory 930) to cause the device 905 to perform various functions (e.g., functions or tasks supporting medical provider to medical provider communications).

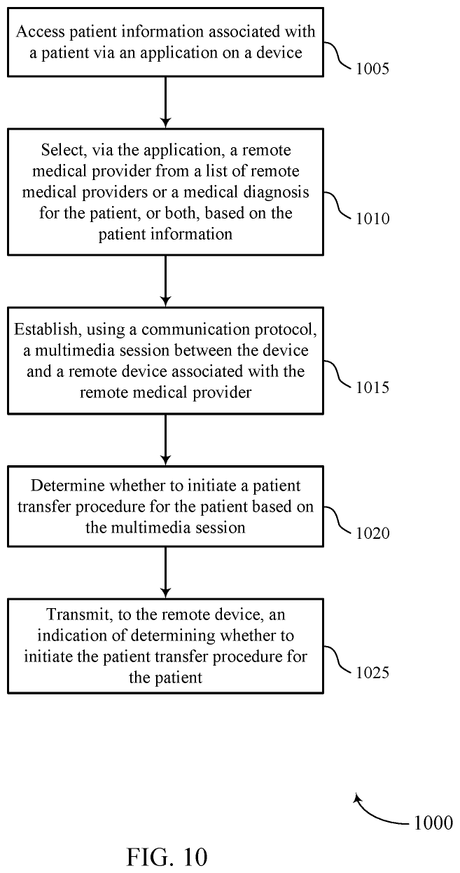

FIG. 10 shows a flowchart illustrating a method 1000 that supports medical provider to medical provider communications in accordance with aspects of the present disclosure. The operations of method 1000 may be implemented by a device or its components as described herein. For example, the operations of method 1000 may be performed by a communications manager as described with reference to FIGS. 6 through 9. In some examples, a device may execute a set of instructions to control the functional elements of the device to perform the functions described below. Additionally or alternatively, a device may perform aspects of the functions described below using special-purpose hardware.

At 1005, the device may access patient information associated with a patient via an application on the device. The operations of 1005 may be performed according to the methods described herein. In some examples, aspects of the operations of 1005 may be performed by an information component as described with reference to FIGS. 6 through 9.

At 1010, the device may select, via the application, a remote medical provider from a list of remote medical providers or a medical diagnosis for the patient, or both, based on the patient information. The operations of 1010 may be performed according to the methods described herein. In some examples, aspects of the operations of 1010 may be performed by a selection component as described with reference to FIGS. 6 through 9.

At 1015, the device may establish, using a communication protocol, a multimedia session between the device and a remote device associated with the remote medical provider. The operations of 1015 may be performed according to the methods described herein. In some examples, aspects of the operations of 1015 may be performed by a session component as described with reference to FIGS. 6 through 9.

At 1020, the device may determine whether to initiate a patient transfer procedure for the patient based on the multimedia session. The operations of 1020 may be performed according to the methods described herein. In some examples, aspects of the operations of 1020 may be performed by a transfer component as described with reference to FIGS. 6 through 9.

At 1025, the device may transmit, to the remote device, an indication of determining whether to initiate the patient transfer procedure for the patient. The operations of 1025 may be performed according to the methods described herein. In some examples, aspects of the operations of 1025 may be performed by a transfer component as described with reference to FIGS. 6 through 9.

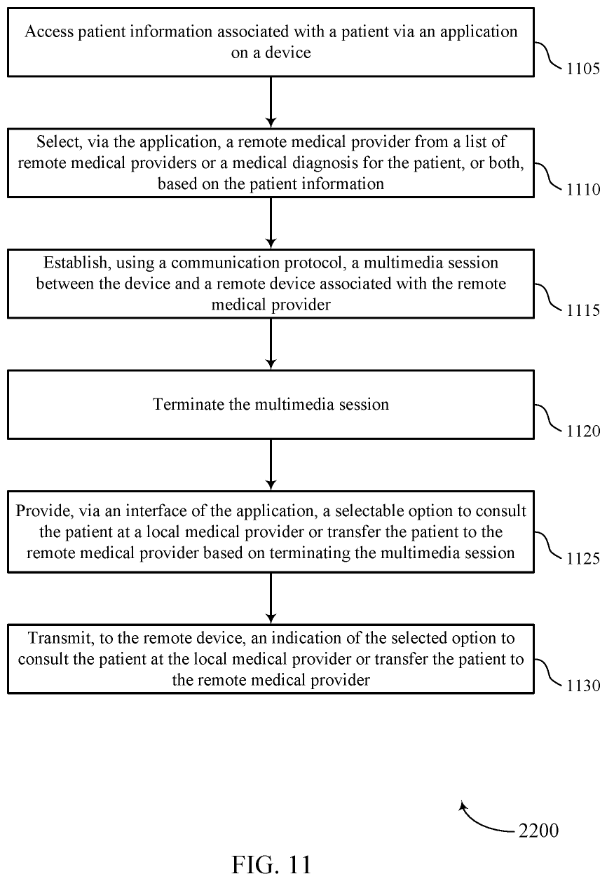

FIG. 11 shows a flowchart illustrating a method 1100 that supports medical provider to medical provider communications in accordance with aspects of the present disclosure. The operations of method 1100 may be implemented by a device or its components as described herein. For example, the operations of method 1100 may be performed by a communications manager as described with reference to FIGS. 6 through 9. In some examples, a device may execute a set of instructions to control the functional elements of the device to perform the functions described below. Additionally or alternatively, a device may perform aspects of the functions described below using special-purpose hardware.

At 1105, the device may access patient information associated with a patient via an application on the device. The operations of 1105 may be performed according to the methods described herein. In some examples, aspects of the operations of 1105 may be performed by an information component as described with reference to FIGS. 6 through 9.

At 1110, the device may select, via the application, a remote medical provider from a list of remote medical providers or a medical diagnosis for the patient, or both, based on the patient information. The operations of 1110 may be performed according to the methods described herein. In some examples, aspects of the operations of 1110 may be performed by a selection component as described with reference to FIGS. 6 through 9.

At 1115, the device may establish, using a communication protocol, a multimedia session between the device and a remote device associated with the remote medical provider. The operations of 1115 may be performed according to the methods described herein. In some examples, aspects of the operations of 1115 may be performed by a session component as described with reference to FIGS. 6 through 9.

At 1120, the device may terminate the multimedia session. The operations of 1120 may be performed according to the methods described herein. In some examples, aspects of the operations of 1120 may be performed by a session component as described with reference to FIGS. 6 through 9.

At 1125, the device may provide, via an interface of the application, a selectable option to consult the patient at a local medical provider or transfer the patient to the remote medical provider based on terminating the multimedia session. The operations of 1125 may be performed according to the methods described herein. In some examples, aspects of the operations of 1125 may be performed by a selection component as described with reference to FIGS. 6 through 9.

At 1130, the device may transmit, to the remote device, an indication of the selected option to consult the patient at the local medical provider or transfer the patient to the remote medical provider. The operations of 1130 may be performed according to the methods described herein. In some examples, aspects of the operations of 1130 may be performed by a transfer component as described with reference to FIGS. 6 through 9.

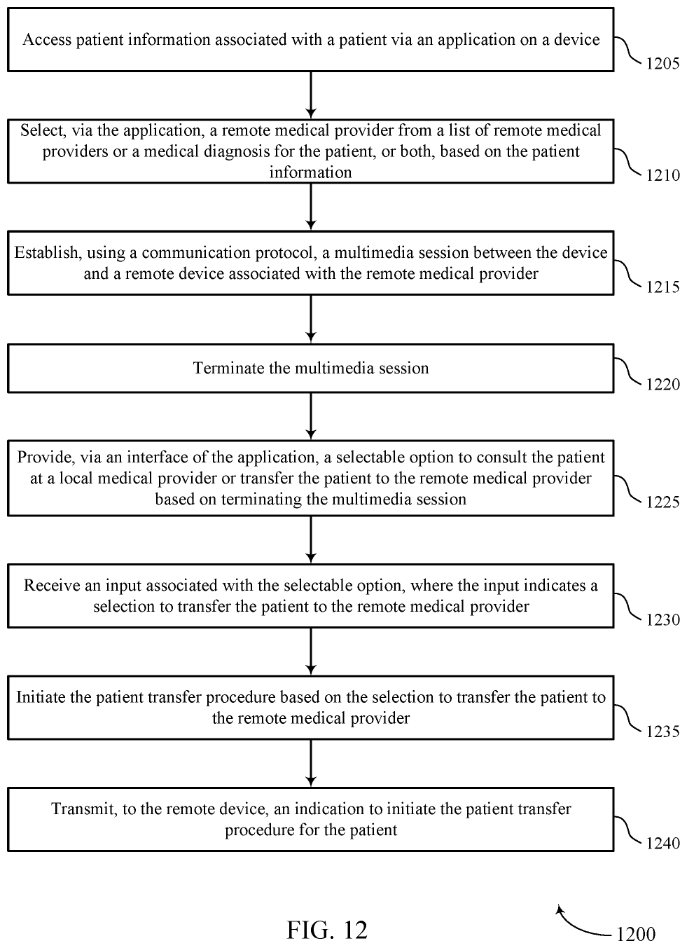

FIG. 12 shows a flowchart illustrating a method 1200 that supports medical provider to medical provider communications in accordance with aspects of the present disclosure. The operations of method 1200 may be implemented by a device or its components as described herein. For example, the operations of method 1200 may be performed by a communications manager as described with reference to FIGS. 6 through 9. In some examples, a device may execute a set of instructions to control the functional elements of the device to perform the functions described below. Additionally or alternatively, a device may perform aspects of the functions described below using special-purpose hardware.

At 1205, the device may access patient information associated with a patient via an application on the device. The operations of 1205 may be performed according to the methods described herein. In some examples, aspects of the operations of 1205 may be performed by an information component as described with reference to FIGS. 6 through 9.

At 1210, the device may select, via the application, a remote medical provider from a list of remote medical providers or a medical diagnosis for the patient, or both, based on the patient information. The operations of 1210 may be performed according to the methods described herein. In some examples, aspects of the operations of 1210 may be performed by a selection component as described with reference to FIGS. 6 through 9.

At 1215, the device may establish, using a communication protocol, a multimedia session between the device and a remote device associated with the remote medical provider. The operations of 1215 may be performed according to the methods described herein. In some examples, aspects of the operations of 1215 may be performed by a session component as described with reference to FIGS. 6 through 9.

At 1220, the device may terminate the multimedia session. The operations of 1220 may be performed according to the methods described herein. In some examples, aspects of the operations of 1220 may be performed by a session component as described with reference to FIGS. 6 through 9.

At 1225, the device may provide, via an interface of the application, a selectable option to consult the patient at a local medical provider or transfer the patient to the remote medical provider based on terminating the multimedia session. The operations of 1225 may be performed according to the methods described herein. In some examples, aspects of the operations of 1225 may be performed by a selection component as described with reference to FIGS. 6 through 9.

At 1230, the device may receive an input associated with the selectable option, where the input indicates a selection to transfer the patient to the remote medical provider. The operations of 1230 may be performed according to the methods described herein. In some examples, aspects of the operations of 1230 may be performed by a selection component as described with reference to FIGS. 6 through 9.

At 1235, the device may initiate the patient transfer procedure based on the selection to transfer the patient to the remote medical provider. The operations of 1235 may be performed according to the methods described herein. In some examples, aspects of the operations of 1235 may be performed by a transfer component as described with reference to FIGS. 6 through 9.

At 1240, the device may transmit, to the remote device, an indication to initiate the patient transfer procedure for the patient. The operations of 1240 may be performed according to the methods described herein. In some examples, aspects of the operations of 1240 may be performed by a transfer component as described with reference to FIGS. 6 through 9.

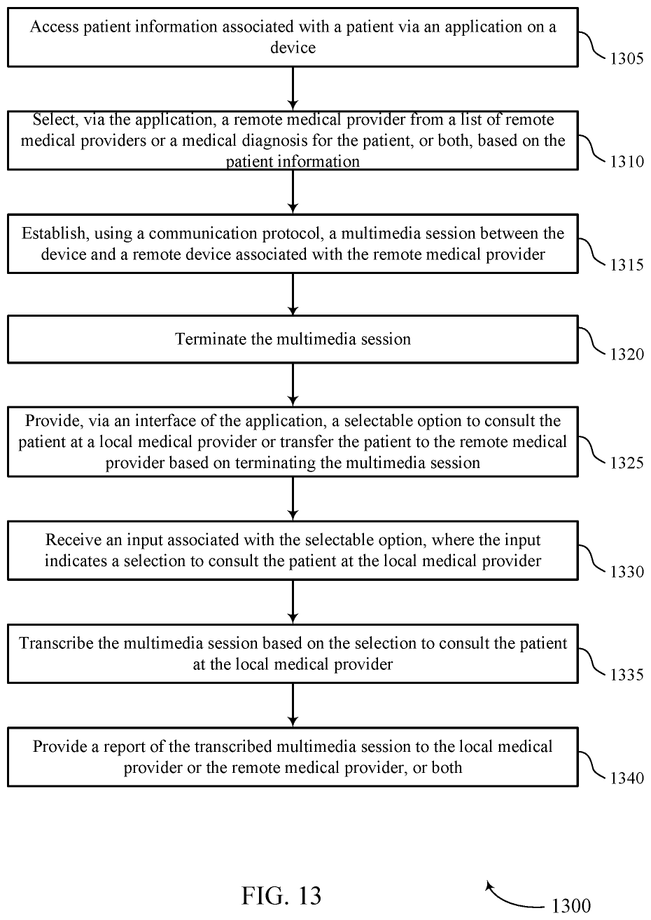

FIG. 13 shows a flowchart illustrating a method 1300 that supports medical provider to medical provider communications in accordance with aspects of the present disclosure. The operations of method 1300 may be implemented by a device or its components as described herein. For example, the operations of method 1300 may be performed by a communications manager as described with reference to FIGS. 6 through 9. In some examples, a device may execute a set of instructions to control the functional elements of the device to perform the functions described below. Additionally or alternatively, a device may perform aspects of the functions described below using special-purpose hardware.

At 1305, the device may access patient information associated with a patient via an application on the device. The operations of 1305 may be performed according to the methods described herein. In some examples, aspects of the operations of 1305 may be performed by an information component as described with reference to FIGS. 6 through 9.

At 1310, the device may select, via the application, a remote medical provider from a list of remote medical providers or a medical diagnosis for the patient, or both, based on the patient information. The operations of 1310 may be performed according to the methods described herein. In some examples, aspects of the operations of 1310 may be performed by a selection component as described with reference to FIGS. 6 through 9.

At 1315, the device may establish, using a communication protocol, a multimedia session between the device and a remote device associated with the remote medical provider. The operations of 1315 may be performed according to the methods described herein. In some examples, aspects of the operations of 1315 may be performed by a session component as described with reference to FIGS. 6 through 9.

At 1320, the device may terminate the multimedia session. The operations of 1320 may be performed according to the methods described herein. In some examples, aspects of the operations of 1320 may be performed by a session component as described with reference to FIGS. 6 through 9.

At 1325, the device may provide, via an interface of the application, a selectable option to consult the patient at a local medical provider or transfer the patient to the remote medical provider based on terminating the multimedia session. The operations of 1325 may be performed according to the methods described herein. In some examples, aspects of the operations of 1325 may be performed by a selection component as described with reference to FIGS. 6 through 9.

At 1330, the device may receive an input associated with the selectable option, where the input indicates a selection to consult the patient at the local medical provider. The operations of 1330 may be performed according to the methods described herein. In some examples, aspects of the operations of 1330 may be performed by a selection component as described with reference to FIGS. 6 through 9.

At 1335, the device may transcribe the multimedia session based on the selection to consult the patient at the local medical provider. The operations of 1335 may be performed according to the methods described herein. In some examples, aspects of the operations of 1335 may be performed by a session component as described with reference to FIGS. 6 through 9.

At 1340, the device may provide a report of the transcribed multimedia session to the local medical provider or the remote medical provider, or both. The operations of 1340 may be performed according to the methods described herein. In some examples, aspects of the operations of 1340 may be performed by a session component as described with reference to FIGS. 6 through 9.

It should be noted that the methods described herein describe possible implementations, and that the operations and the steps may be rearranged or otherwise modified and that other implementations are possible. Further, aspects from two or more of the methods may be combined.

Although aspects of an LTE, LTE-A, LTE-A Pro, or NR system may be described for purposes of example, the techniques described herein are applicable beyond LTE, LTE-A, LTE-A Pro, or NR networks. For example, the described techniques may be applicable to various other wireless communications systems such as Ultra Mobile Broadband (UMB), Institute of Electrical and Electronics Engineers (IEEE) 802.11 (Wi-Fi), IEEE 802.16 (WiMAX), IEEE 802.20, Flash-OFDM, as well as other systems and radio technologies not explicitly mentioned herein.

Information and signals described herein may be represented using any of a variety of different technologies and techniques. For example, data, instructions, commands, information, signals, bits, symbols, and chips that may be referenced throughout the description may be represented by voltages, currents, electromagnetic waves, magnetic fields or particles, optical fields or particles, or any combination thereof.

The various illustrative blocks and components described in connection with the disclosure herein may be implemented or performed with a general-purpose processor, a DSP, an ASIC, a CPU, an FPGA or other programmable logic device, discrete gate or transistor logic, discrete hardware components, or any combination thereof designed to perform the functions described herein. A general-purpose processor may be a microprocessor, but in the alternative, the processor may be any processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described herein may be implemented using software executed by a processor, hardware, firmware, hardwiring, or combinations of any of these. Features implementing functions may also be physically located at various positions, including being distributed such that portions of functions are implemented at different physical locations.

Computer-readable media includes both non-transitory computer storage media and communication media including any medium that facilitates transfer of a computer program from one place to another. A non-transitory storage medium may be any available medium that may be accessed by a general-purpose or special purpose computer. By way of example, and not limitation, non-transitory computer-readable media may include random-access memory (RAM), read-only memory (ROM), electrically erasable programmable ROM (EEPROM), flash memory, compact disk (CD) ROM or other optical disk storage, magnetic disk storage or other magnetic storage devices, or any other non-transitory medium that may be used to carry or store desired program code means in the form of instructions or data structures and that may be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of computer-readable medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

As used herein, including in the claims, “or” as used in a list of items (e.g., a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an example step that is described as “based on condition A” may be based on both a condition A and a condition B without departing from the scope of the present disclosure. In other words, as used herein, the phrase “based on” shall be construed in the same manner as the phrase “based at least in part on.”

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label, or other subsequent reference label.

The description set forth herein, in connection with the appended drawings, describes example configurations and does not represent all the examples that may be implemented or that are within the scope of the claims. The term “example” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, known structures and devices are shown in block diagram form in order to avoid obscuring the concepts of the described examples.

The description herein is provided to enable a person having ordinary skill in the art to make or use the disclosure. Various modifications to the disclosure will be apparent to a person having ordinary skill in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for communications, comprising:
accessing patient information associated with a patient via an application on a device;
selecting, via the application, a remote medical provider from a list of remote medical providers or a medical diagnosis for the patient, or both, based at least in part on the patient information;
establishing, using a communication protocol, a multimedia session between the device and a remote device associated with the remote medical provider;
determining whether to initiate a patient transfer procedure for the patient based at least in part on the multimedia session; and
transmitting, to the remote device, an indication of determining whether to initiate the patient transfer procedure for the patient.

2. The method of claim 1, further comprising:
terminating the multimedia session; and
providing, via an interface of the application, a selectable option to consult the patient at a local medical provider or transfer the patient to the remote medical provider based at least in part on terminating the multimedia session.

3. The method of claim 2, further comprising:
receiving an input associated with the selectable option, wherein the input indicates a selection to transfer the patient to the remote medical provider; and
initiating the patient transfer procedure based at least in part on the selection to transfer the patient to the remote medical provider,
wherein transmitting, to the remote device, the indication is based at least in part on the initiating.

4. The method of claim 3, wherein the patient transfer procedure comprises:
transmitting a patient transfer notification to the remote device associated with the remote medical provider, the patient transfer notification including the patient information; and
receiving, at the device, an acknowledgment to transfer the patient to the remote medical provider based at least in part on the patient transfer notification.

5. The method of claim 4, further comprising:
receiving, from the remote device associated with the remote medical provider, patient transfer information comprising physician information, room information, additional medical personnel information, or any combination thereof.

6. The method of claim 2, further comprising:
receiving an input associated with the selectable option, wherein the input indicates a selection to consult the patient at the local medical provider;
transcribing the multimedia session based at least in part on the selection to consult the patient at the local medical provider; and
providing a report of the transcribed multimedia session to the local medical provider or the remote medical provider, or both.

7. The method of claim 1, further comprising:
transmitting, to the remote device associated with the remote medical provider, a request message to establish the multimedia session, the request message comprising session information for the multimedia session; and
receiving, from the remote device associated with the remote medical provider, a response message based at least in part on the request message, the response message comprising a second indication of accepting, delaying, or declining the request message to establish the multimedia session, the session information comprising the patient information or the medical diagnosis for the patient, or both,
wherein establishing the multimedia session between the device and the remote device is based at least in part on the response message.

8. The method of claim 7, further comprising:
retrieving one or more images corresponding to the medical diagnosis for the patient; and
including, in the session information, the one or more images corresponding to the medical diagnosis for the patient.

9. The method of claim 7, wherein the multimedia session comprises an audio session or a video session, or both.

10. The method of claim 1, further comprising:
determining the list of remote medical providers based at least in part on the patient information or the medical diagnosis for the patient, or both; and
determining one or more locations associated with one or more remote medical providers, including the remote medical provider, in the list of remote medical providers that satisfy a location threshold relative to the patient,
wherein selecting the remote medical provider from the list of remote medical providers is based at least in part on the location threshold relative to the patient.

11. The method of claim 1, further comprising:
determining the medical diagnosis for the patient based at least in part on the patient information; and
including a second indication of the medical diagnosis in session information for the multimedia session.

12. The method of claim 1, further comprising:
identifying a patient account corresponding to the patient, wherein accessing the patient information comprises:
retrieving the patient information from the patient account via the application running on the device or a server, or any combination thereof.

13. The method of claim 1, further comprising:
capturing an image of a document including the patient information via a camera of the device;
determining one or more information elements of the document using an optical character recognition operation on the document; and
compiling the patient information based at least in part on the one or more information elements.

14. The method of claim 13, further comprising:
generating a patient account corresponding to the patient associated with the patient information; and
storing the patient account in a database remote or local to the device or a server, or any combination thereof, wherein accessing the patient information comprises:
retrieving the patient information from the patient account via the application running on the device or the server, or any combination thereof.

15. The method of claim 1, further comprising:
determining a request to access the application running on the device; and
providing the access to the application running on the device based at least in part on an authentication procedure.

16. The method of claim 15, wherein the authentication procedure comprises:
receiving credential information via the device, the credential information comprising a username and a password, a facial recognition, a voice recognition, a biosignature recognition, or any combination thereof; and
authenticating the device based at least in part on the credential information.

17. The method of claim 15, wherein the authentication procedure comprises a two-factor authentication procedure comprising:
receiving first credential information from via the device;
authenticating the first credential information;
receiving second credential information via the device based at least in part on authenticating the first credential information; and
authenticating the second credential information, wherein the first credential information or the second credential information, or both comprises a username and a password, a facial recognition, a voice recognition, a biosignature recognition, or any combination thereof.

18. The method of claim 1, wherein the medical diagnosis for the patient comprises a cardiological medical diagnosis, a neurological medical diagnosis, a trauma medical diagnosis, or a triage medical diagnosis, or any combination thereof.

19. An apparatus for communications, comprising:
a processor,
memory coupled with the processor; and
instructions stored in the memory and executable by the processor to cause the apparatus to:
access patient information associated with a patient via an application on the apparatus;
select, via the application, a remote medical provider from a list of remote medical providers or a medical diagnosis for the patient, or both, based at least in part on the patient information;
establish, using a communication protocol, a multimedia session between the apparatus and a remote apparatus associated with the remote medical provider;
determine whether to initiate a patient transfer procedure for the patient based at least in part on the multimedia session; and
transmit, to the remote apparatus, an indication of determining whether to initiate the patient transfer procedure for the patient.

20. An apparatus for communications, comprising:
means for accessing patient information associated with a patient via an application on the apparatus;
means for selecting, via the application, a remote medical provider from a list of remote medical providers or a medical diagnosis for the patient, or both, based at least in part on the patient information;
means for establishing, using a communication protocol, a multimedia session between the apparatus and a remote apparatus associated with the remote medical provider;
means for determining whether to initiate a patient transfer procedure for the patient based at least in part on the multimedia session; and
means for transmitting, to the remote apparatus, an indication of determining whether to initiate the patient transfer procedure for the patient.
